(19) United States
(12) Patent Application Publication
(10) Pub. No.: US 2022/0005548 A1
(43) Pub. Date: Jan. 6, 2022

(54) INTELLIGENT SYSTEM AND METHODS FOR THERAPEUTIC TARGET IDENTIFICATION

(71) Applicant: I2DX, INC., San Francisco, CA (US)

(72) Inventors: Janos REDEI, San Francisco, CA (US); Arthur MIKHNO, Princeton, NJ (US)

(73) Assignee: I2DX, INC., San Francisco, CA (US)

(21) Appl. No.: 17/289,695

(22) PCT Filed: Oct. 31, 2019
(86) PCT No.: PCT/US2019/059138

§ 371 Date: Apr. 28, 2021

Related U.S. Application Data
(60) Provisional application No. 62/754,481, filed on Nov. 1, 2018.

(51) Int. Cl. G16B 25/00; G16B 20/00; G06T 7/00; G06N 20/20; G06N 3/08

(52) U.S. Cl. CPC G16B 25/00; G16B 20/00; G06T 7/0012; G06N 20/20; G06T 2207/10108; G06T 2207/10088; G06T 2207/20084; G06T 2207/20081; G06T 2207/10104; G06N 3/08

(57) ABSTRACT

A system and computer-implemented method is provided for uncovering genetic drivers of disease in neurodegeneration and other central nervous system (CNS) diseases for novel therapeutic target identification. The process can start with a first integrated, quantitative “deep” imaging phenotype, that accurately reflects disease at a given time-point (cross-sectionally) and returns candidate single nucleotide polymorphisms (SNPs) and/or genes. The candidate SNPs/genes are further validated, by using a second machine learning and/or artificial intelligence (AD-based image analysis operation to assess clinical response, and may include gene expression profiling, and target plausibility analysis including pathway mapping. A set of candidate SNPs/genes may be constructed to accurately predict the first imaging phenotype with a deep learning model from said SNPs/genes to serve as an additional validation step.

### BACKGROUND

Neurodegenerative and other central nervous system (CNS) diseases are in need of novel targets for therapeutic development, particularly as the field is moving away from symptomatic approaches towards disease modification and prevention. Uncovering and targeting novel genetic drivers of disease is of high interest in Alzheimer's disease (AD) as nearly all clinical trials to date have failed.

Artificial Intelligence (AI) and machine learning-based approaches are now emerging to aid pharmaceutical drug discovery and development to improve efficiency. The industry is attempting to leverage “big data” sources such as electronic health record (EHR) data by applying, for example, deep learning for pharmaceutical drug discovery and development. Such “big data” sources, however, may be of varying quality. Moreover, predictions made by the AI may be difficult to explain and/or erroneous. Therefore, there is a trend towards what is called xAI (explainable AI), where the patterns detected and predictions made are transparent and plausible. “Good” (e.g., clean) data sources are further hard to come by and need “few shot learning” approaches to achieve utility; this is in stark contrast to certain deep learning approaches that may require thousands or millions of training cases. Few shot learning systems have been likened to how toddlers learn a new task, with far less training cases.

Thus, there is an unmet need for transparent, intelligent systems that can extract knowledge from limited data sources to aid biopharmaceutical research and development, and to decode novel genetic drivers of disease, in CNS disease and beyond.

### SUMMARY

The disclosure describes an efficient system and computer-implemented method for uncovering genetic drivers of disease in neurodegeneration and other central nervous system (CNS) diseases, from limited amounts of human data, for novel therapeutic target identification. According to an aspect of an embodiment, the disclosure describes a process starting with a first integrated, quantitative “deep” imaging phenotype, that accurately reflects disease at a given time-point (cross-sectionally) and returns candidate single nucleotide polymorphisms (SNPs) and/or genes. The candidate SNPs/genes are further validated, at least in part by using a second machine learning and/or artificial intelligence (AI)-based image analysis operation to assess clinical response, and may further be followed by gene expression profiling, and target plausibility analysis including pathway mapping. A set of candidate SNPs/genes may be constructed to accurately predict the first imaging phenotype with a deep learning model from said SNPs/genes to serve as an additional validation step.

Example embodiments of the present disclosure provide computer techniques or use in studying Alzheimer's disease in order to discover therapeutic targets with just a few hundred samples, and further achieve performance on par with other gene discovery/risk prediction approaches that require thousands of samples.

Now, some embodiments provide an improvement within the field of imaging-genetics and AI, in so far as in one embodiment, said computer-implemented method starts with said first integrated, quantitative “deep” imaging phenotype, that accurately reflects disease at a given time-point (i.e., cross-sectionally) within a single measure, and returns candidate single nucleotide polymorphisms (SNPs) and/or genes, for example by performing a quantitative trait locus genome-wide association study (QTL GWAS).

Other QTL GWAS approaches to date have for example, utilized quantitative measures typically found in a medical chart or EHR. Example quantitative measures used in prior approaches may include noisy and discrete cognitive testing result, or a measure of global Amyloid, or perhaps Tau positivity status (e.g., by cerebrospinal fluid (CSF)). These prior approaches have had limited success at best, mostly due to a lack of accuracy to predict, for example, conversion to AD from early disease stages, among other limitations. In contrast to QTL GWAS studies, case-control studies can be confounded by uncertainty as to what constitutes an AD “case”, for many reasons including that these case-control studies sometimes rely on informal family history information. A newer but still limited approach for target discovery is aggregation of brain bank samples and analysis thereof by, for example RNAseq transcriptomics. While brain bank data may be a valuable source for target validation (confirmation), it is yet to uncover by itself meaningful novel therapeutic targets in AD, in part because of possible noise due to sample preparation and time of sample capture after death.

In contrast to these and other prior approaches, molecular multi-modal imaging, such as voxel-based quantitation of positron emission tomography (PET) and magnetic resonance imaging (MRI) is more suitable, as it allows to encode local spatial information and capture molecular events in vivo.

In some embodiments, a computer-implemented method of identifying genetic biomarkers of a condition can include: performing a quantitative image analysis of an image of a phenotype for a condition of a subject for a plurality of subjects with the condition to obtain a first accurate quantitative phenotype; performing a quantitative genome analysis on each subject of the plurality of subjects or a plurality of different subjects; obtaining at least one candidate genetic biomarker for the condition in the plurality of subjects from the quantitative image analysis and the quantitative genome analysis; predicting a clinical response against the at least one candidate genetic biomarker to validate the at least one candidate genetic biomarker; identifying at least one therapeutic target for the condition based on the at least one candidate genetic biomarker, wherein the at least one therapeutic target for the condition is biologically associated with the at least one candidate genetic biomarker; and generating a report with the identified at least one therapeutic target for the condition. In some aspects, the performing a quantitative genome analysis includes performing a quantitative trait locus genome-wide association study (QTL GWAS).

In some embodiments, the methods can include determining a validation metric by predicting a clinical response against the at least one candidate genetic biomarker to validate the at least one candidate genetic biomarker. In some aspects, the clinical response prediction is performed to modulate at least one biological pathway associated with the at least one genetic biomarker. In some aspects, the predicted clinical response is performed to inhibit at least one biologically active protein of a biological pathway associated with the at least one genetic biomarker.

In some embodiments, the method can include at least one of: obtaining the images of the phenotype of the subjects, wherein the images accurately illustrate the condition at a given time-point; obtaining a phenotype accuracy of greater than or about 85% for a quantitative phenotype; detecting a disease as the condition from the images of the phenotype of the subjects; defining disease activity across a disease spectrum; or performing a standardized uptake value ratio (SUVR) analysis with the images of the phenotype of the subject. In some embodiments, the performing of the quantitative image analysis of the image of the phenotype includes at least one of: calculating a voxel-based amyloid standardized uptake value ratio (SUVR) from the images; calculating a voxel-based dopamine transporter single photon emission computed tomography (DAT-SPECT) quantitation from the images; calculating a voxel-based alpha-synuclein ligand binding from the images; or calculating a regional serotonin receptor ligand binding from the images.

In some embodiments, the images are selected from positron emission tomography (PET) and magnetic resonance imaging (MRI), single photon emission computed tomography (SPECT), and combinations thereof.

In some embodiments, the predicting of the clinical response includes at least one of: performing a hippocampus-masked voxel-based Tau SUVR by genotype quantitation; performing a determination of memory tracking by a voxel-based Tau SUVR by genotype quantitation with the hippocampus; performing a voxel-based Tau quantitation within the hippocampus; performing a Tau imaging analysis that tracks a clinical response; or performing a clinical response tracking measure. In some aspects, the predicting of the clinical response includes at least one of: performing a tremor quantitation; or performing an automated tremor quantitation, wherein the tremor quantitation is optionally by genotype. In some aspects, the predicting of the clinical response includes calculating a voxel-based serotonin receptor ligand binding from images.

In some embodiments, the methods can include performing a phenotype prediction from a genotype based on the at least one candidate genetic biomarker for the condition. In some aspects, the phenotype prediction includes at least one of: phenotype prediction with deep learning from a single nucleotide polymorphism (SNP) model; a quantitative phenotype analysis with positron emission tomography (PET) images; predict voxel-based amyloid SUVRs with deep learning SNP model; predict voxel-based DAT-SPECT quantitation with deep learning SNP model; predict voxel-based alpha-synuclein ligand binding with deep learning SNP model; or predict regional serotonin receptor ligand binding with deep learning SNP model.

In some embodiments, the method can include at least one of: listing the at least one candidate genetic biomarker in the report; analyzing a biological pathway having the at least one candidate genetic biomarker; or comparing the at least one candidate genetic biomarker to a biological pathway associated with the condition.

In some embodiments, the condition is: a syndrome having a set of medical signs and symptoms that are correlated with each other in the subject; a disease having a pathophysiological response to external or internal factors in the subject; or a disorder having a disruption to regular bodily structure and/or function in the subject. In some aspects, the condition is selected from Alzheimer's disease, Parkinson's disease, and Major depressive disorder (MDD).

In some embodiments, a computer-implemented method can include: performing an accurate quantitative phenotype analysis; performing a quantitative trait locus genome-wide association study (QTL GWAS); obtaining at least one candidate genetic biomarker for the condition in the plurality of subjects from the quantitative image analysis and the quantitative genome analysis; predicting a clinical response against the at least one candidate genetic biomarker to validate the at least one candidate genetic biomarker; and performing a phenotype prediction with deep learning SNP model.

In some embodiments, a computer-implemented method can include: calculating voxel-based DAT-SPECT quantitation from SPECT and T1 MRI images; performing a quantitative trait locus genome-wide association study (QTL GWAS); obtaining at least one candidate genetic biomarker for the condition in the plurality of subjects from the quantitative image analysis and the quantitative genome analysis; comparing tremor quantitation by genotype; and predicting voxel-based DAT-SPECT quantitation with deep learning SNP model.

In some embodiments, a computer-implemented method can include: calculating voxel-based alpha-synuclein ligand binding from PET and T1 MRI images; performing a quantitative trait locus genome-wide association study (QTL GWAS); obtaining at least one candidate genetic biomarker for the condition in the plurality of subjects from the quantitative image analysis and the quantitative genome analysis; comparing tremor quantitation by genotype; and predicting voxel-based alpha-synuclein ligand binding with deep learning SNP model.

In some embodiments, a computer-implemented method can include: calculating regional serotonin receptor ligand binding from PET and T1 MRI images; performing a quantitative trait locus genome-wide association study (QTL GWAS); obtaining at least one candidate genetic biomarker for the condition in the plurality of subjects from the quantitative image analysis and the quantitative genome analysis; calculating voxel-based serotonin receptor ligand binding with PET and T1 MRI images; and predicting regional serotonin receptor ligand binding with deep learning SNP model.

In some embodiments, a computer-implemented method can include: calculating voxel-based amyloid SUVRs from PET and T1 MRI images; performing a quantitative trait locus genome-wide association study (QTL GWAS); obtaining at least one candidate genetic biomarker for the condition in the plurality of subjects from the quantitative image analysis and the quantitative genome analysis; comparing hippocampus-masked voxel-based Tau SUVRs by genotype; predicting voxel-based amyloid SUVRs with deep learning SNP model; and generating a report with the predicted voxel-based amyloid SUVRs.

In some embodiments, a computer-implemented method of training an artificial neural network for detection of Amyloid positivity or early Alzheimer's Disease can include: collecting a set of corresponding positron emission tomography (PET) images, magnetic resonance imaging (MRI) images and corresponding genotype data from a database; calculating voxel-based amyloid SUVRs from the PET images and T1 MRI images to obtain an accurate quantitative phenotype of early Alzheimer's Disease; creating a training set comprising said quantitative phenotype and corresponding genotype data; and training the artificial neural network to predict the quantitative phenotype from genotype data using the training set. In some aspects, the artificial neural network is trained using genotype data comprising at least SNP id 1-32, or a subset of SNP id 1-32, and optionally SNP id 33 of Table 1 herein to predict the quantitative phenotype. In some aspects, the training can include a subset with a sufficient number of SNPs in order to provide the training in order to operate with the methods described herein. The subset may include at least 10 SNPs, at least 15 SNPs, at least 20 SNPs, at least 25 SNPs, or at least 30 SNPs of SNP id 1-32. However, it should be recognized that a higher number of SNPs in the subset may result in better performance.

In some embodiments, a system for identifying genetic biomarkers of a condition can include: a deep phenotyping unit; a QTL GWAS analysis unit downstream from the deep phenotyping unit; and a clinical response validation unit downstream from the QTL GWAS analysis unit. In some aspects, the system includes a gene expression analysis unit downstream from the clinical response validation unit. In some aspects, the system includes a pathway mapping unit downstream from the clinical response validation unit, optionally also downstream from the gene expression analysis unit. In some aspects, the system includes a phenotype prediction unit downstream from the clinical response validation unit, optionally also downstream from the gene expression analysis unit, and optionally also downstream from the pathway mapping unit. In some aspects, the system includes a toolset system, which may include one or more units selected from: a pattern and/or trend analysis unit; a statistical analysis unit; a text mining plausibility analysis unit; a new chemical entity (NCE) prediction unit; or a reporting unit.

In some embodiments, the system can be configured in accordance with one or more of the following. In some aspects, the deep phenotyping unit is configured for performing at least one of: performing an accurate quantitative phenotype analysis; calculating voxel-based amyloid SUVRs from PET and T1 MRI images; calculating voxel-based DAT-SPECT quantitation from SPECT and T1 MRI images; calculating voxel-based alpha-synuclein ligand binding from PET and T1 MRI images; or calculating regional serotonin receptor ligand binding from PET and T1 MRI images. In some aspects, the QTL GWAS analysis unit is configured for performing a quantitative trait locus genome-wide association study (QTL GWAS). In some aspects, the clinical response validation unit is configured for performing at least one of: predicting a clinical response against the at least one candidate genetic biomarker to validate the at least one candidate genetic biomarker; comparing hippocampus-masked voxel-based Tau SUVRs by genotype; comparing tremor quantitation by genotype; or calculating voxel-based serotonin receptor ligand binding with PET and T1 MRI images.

In some embodiments, the system can include one or more of: a gene expression analysis unit configured for performing gene expression profiling, which may or may not be automated; a pathway mapping unit configured for mapping one or more biological pathways of genes identified by the gene expression analysis unit; a phenotype prediction unit configured to use a deep learning model to predict the phenotypes identified by the deep phenotyping unit or to predict new phenotypes for new investigations of new conditions; a pattern and/or trend analysis unit optionally used with the deep phenotyping unit and configured to determine patterns or trends for the phenotype; a statistical analysis unit configured to perform statistical analyses of any data or metric of the methods; a text mining plausibility analysis unit configured to determine therapeutic target plausibility by text mining and/or to determine a chemical modulator of a biological entity of a biological pathway associated with the therapeutic target; a new chemical entity (NCE) prediction unit configured to design or generate an NCE that can modulate the therapeutic target; or a reporting unit is configured to generate reports.

### DESCRIPTION OF THE DRAWINGS

Embodiments of the disclosure are illustrated by way of example, and not by way of limitation, so the disclosure can be better understood, by referring to the following drawings, in which:

FIG. 1: is an example of a flowchart of an efficient computer-implemented method for uncovering genetic drivers of disease in neurodegeneration and other CNS diseases, from far less human data, for novel therapeutic target identification.

FIG. 2: is an example of a flowchart of an efficient computer-implemented method for uncovering genetic drivers of disease in Alzheimer's disease, from far less human data, for novel therapeutic target identification.

FIG. 3: is an example of a flowchart of a computer-implemented method for identifying genetic drivers of disease in Parkinson's disease, using a voxel-based dopamine transporter single photon emission computed tomography (DAT-SPECT) deep imaging phenotype calculated from DAT-SPECT and MRI, and automated tremor quantitation for clinical response assessment.

FIG. 4: is an example of a flowchart of a computer-implemented method for identifying genetic drivers of disease in Parkinson's disease, using a voxel-based deep imaging phenotype calculated from alpha-synuclein PET and MRI, and automated tremor quantitation for clinical response assessment.

FIG. 5: is an example of a flowchart of a computer-implemented method for identifying genetic drivers of disease in Major depressive disorder (MDD), using a deep imaging phenotype calculated from regional serotonin receptor ligand binding from PET and T1 MRI, and a Voxel-based serotonin receptor ligand binding from PET and T1 MRI for clinical response assessment.

FIG. 6: is a diagram illustrating the major components, and the data flow of an example intelligent system for therapeutic target identification in drug discovery.

FIG. 7: is a diagram demonstrating high (>/=85%) accuracy according to the new National Institute on Aging and Alzheimer's Association (NIA-AA) “ATN” (beta amyloid deposition, pathologic tau, and neurodegeneration) criteria, of a quantitative “deep” phenotype for target identification, here by way of example, a voxel-based standardized uptake value ratio (SUVR) calculated from a single Amyloid PET and T1 MRI scan, demonstrating discrimination between different ATN biomarker profile groups.

FIG. 8: is a diagram demonstrating preliminary results in 30 subjects for a voxel-based Tau PET quantitation within the hippocampus to assess clinical response, which showed a stronger correlation with mini-mental state examination (MMSE) than reported by other investigators, as it relates to either Tau PET quantitation, or Amyloid PET quantitation.

FIG. 9: is an example of a report generated by a system for uncovering genetic drivers of disease in Alzheimer's disease, here by way of example the candidate Lysophosphatidylcholine Acyltransferase 2 (LPCAT2) gene/protein; a suggestive association for protective SNP rs4402561 has been detected with a first accurate quantitative phenotype (PETQ) and the step-down pattern confirmed (trailing p=0.054) by a second clinical response tracking measure (PETQ TAU).

FIG. 10: is an example of a report generated by a system for uncovering genetic drivers of disease in Alzheimer's disease, here by way of example the candidate LPCAT2 gene/protein; blood expression profiling confirms a highly significant step-up pattern of a LPCAT2 probe possibly associated with a regulatory transcript.

FIG. 11: is an example of a report generated by a system for uncovering genetic drivers of disease in Alzheimer's disease, here by way of example the candidate LPCAT2 gene/protein; brain bank gene expression profiling confirms a trending step-up pattern of a LPCAT2 probe between young controls (yCTR), age-matched older controls (oCTR) and Alzheimer's disease subjects (AD), within the superior frontal cortex.

FIG. 12: is an example of a report generated by a system for uncovering genetic drivers of disease in Alzheimer's disease, here by way of example for the candidate LPCAT2 gene/protein; blood expression profiling of pro-inflammatory cytokine IL6 by rs4402561 minor allele carrier status confirms a trending step-down pattern and explaining protection.

FIG. 13: is a diagram illustrating the major components, and the data flow in an example phenotype predictor (DeepQnt) within the intelligent system for therapeutic target identification in drug discovery; a set of candidate SNPs/genes may be constructed to predict the original imaging phenotype with a deep learning model from discovered SNPs/genes and may serve as additional validation step (VCF file is shown, but BCF file may also be used).

FIG. 14: is a diagram demonstrating phenotype (PETQ SUVR) prediction from genotype with a deep learning based system (DeepQnt) within the intelligent system for therapeutic target identification in drug discovery. Subjects that have an Amyloid positive PET scan (assessed by an FDA approved method, SPAP) are denoted SPAP+.

FIG. 15: is a diagram demonstrating the performance of an example phenotype predictor (DeepQnt) within the intelligent system for therapeutic target identification in drug discovery; by way of example, the powerful computer technique in Alzheimer's Disease (AD) allows to discover therapeutic targets with just a few hundred samples, and achieve comparable performance on par with other gene discovery/risk prediction approaches that require thousands of samples; SPAPpos denotes an Amyloid positive PET scan, NC/C conversion to AD from mild cognitive impairment (MCI) in a dataset with long (>=5 years) follow-up.

FIG. 16: is an example computing device 1600 (e.g., a computer) that may be arranged in some embodiments to perform the methods (or portions thereof) described herein.

FIG. 17: is a diagram demonstrating head-to-head performance comparison of an example phenotype predictor (DeepQnt) within the intelligent system for therapeutic target identification in drug discovery, against a benchmark method (polygenic hazard score, PHS; Desikan et al. PLoS Medicine 2017); by way of example, the disclosed computer technique in Alzheimer's Disease (AD) allows to discover therapeutic targets with just a few hundred samples, and achieve comparable performance (here for detection conversion from MCI to AD) on par with other gene discovery/risk prediction approaches such as PHS and derived from thousands of subjects. NC/C denotes conversion to AD from mild cognitive impairment (MCI) in a dataset with long (>=5 years) follow-up.

FIG. 18: is a diagram demonstrating head-to-head performance comparison of an example phenotype predictor (DeepQnt) within the intelligent system for therapeutic target identification in drug discovery, against a benchmark method (polygenic hazard score, PHS; Desikan et al. PLoS Medicine 2017); by way of example, the disclosed computer technique in Alzheimer's Disease (AD) allows to discover therapeutic targets with just a few hundred samples, and achieve comparable performance (here for detection of Amyloid positivity in MCI subjects). SPAP positivity denotes Amyloid positive PET scan; MCI, mild cognitive impairment.

FIG. 19: is a diagram demonstrating performance comparison of an example phenotype predictor (DeepQnt) within the intelligent system for therapeutic target identification in drug discovery, for 54 MCI test cases; by way of example, the disclosed computer technique in Alzheimer's Disease (AD) allows to discover therapeutic targets with just a few hundred samples, and achieve increased performance (here for detection conversion from MCI to AD) as compared to APOE4 carrier status alone. MCI, denotes mild cognitive impairment in a dataset with long (>=5 years) follow-up.

### DETAILED DESCRIPTION

Generally, the present technology relates to a method of processing data to identify genetic information that is indicative of a condition, such as a disorder, disease, or syndrome as well as a system for implementing the method. The protocols described herein can be used for identifying SNPs or other genotype information that is indicative of the condition. The method provides metrics that can be used to analyze and process data sets for a population of subjects so that the genotypes can be identified that are related to the phenotypes of a condition.

In some embodiments, the protocols can use a data point in time of a disease, which is referred to as a cross-sectional analysis. However, the protocols can use multiple data points over time of a disease, which is referred to as longitudinal analysis.

The protocols can analyze the data points across a population of subjects for a quantitative measure of the phenotype of the subjects. The cross-sectional quantitative measure of a disease at the time point can be analyzed for the phenotype of the condition of the subjects. The cross-sectional measurement can occur very early in the disease, such as in the initial stages even before symptom onset, which can be referred to as preclinical or presymptomatic stages, or in the symptomatic stages.

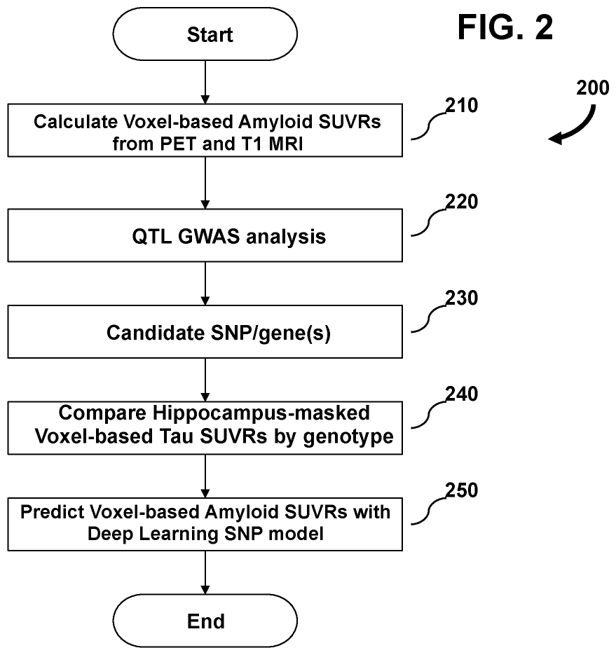

The dataset can have measurements for the condition state that can be analyzed to identify a quantitative phenotype as a metric of the condition state. The new ATN definition as provided herein can be used to determine the condition state. For example, for Alzheimer's disease (AD), the amyloid and Tau positivity can be used to define the preclinical AD state, or the prodromal state, both of which are prior to the full dementia stage. The cross-sectional measure is applied to a data set (e.g., example 1) of subjects with no or some mental deficit, but without full disease state. The metric is applied, such as shown in FIG. 2 in Step 210, and it is determined that the condition state can be accurately identified with the metric, such as with an accuracy of greater than or about 85% accuracy (e.g., defined as accurate quantitative phenotype).

In preclinical controls (i.e., subjects without any symptoms), subjects can also be analyzed with the metric to identify the condition state. The subjects with the potential to develop the condition can therefore be differentiated from subjects not likely to develop the condition. Once the metric is identified, it can be used to predict the potential for a subject to develop the condition, with up to 100% accuracy.

The deep phenotype, as described herein, allows distilling the data down to a metric (e.g., which can be the accurate quantitative phenotype) that results in identifying the disease. The metric also allows for determinations across a spectrum of the condition from a state prior to symptom onset, through onset, and to the full condition state (e.g., Alzheimer's disease).

Once the metric is identified, it can be used with a discovery dataset for validation in subjects that are not specifically linked to the condition. The metric can be processed with the discovery dataset (e.g., one time point per patient, cross-sectional, but time point of one subject can be different from time point of other subject) to identify the genotype of the condition. The protocol thereby matches the genotype to the phenotype in order to identify genetic code linked to the condition across the spectrum of the condition. The genetic analysis can then provide an identification of one or more genes, which may include SNPs, indicative of the condition. This can include the deep phenotype, which can be used for a range of subjects, for diagnostic purposes for susceptibility or having the condition.

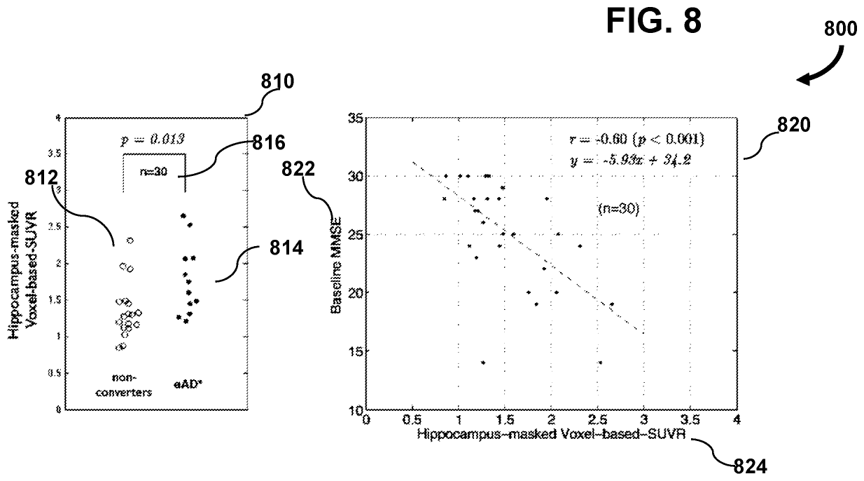

In some embodiments, voxel-based SUVRs can be analyzed by genotype to obtain a voxel metric. For example, Step 240 of FIG. 2 can be used to determine the voxel metric. A new voxel metric can be used to analyze the subject, such as with the hippocampus, which deals with memory. The hippocampus may be used because of the linking with Alzheimer's disease, such as the Tau. The voxel metric is correlated with memory, such as by MMSE as a cognitive measure, which can be shown in FIG. 8 as described herein, which shows the voxel metric tracks memory, a clinical response measure.

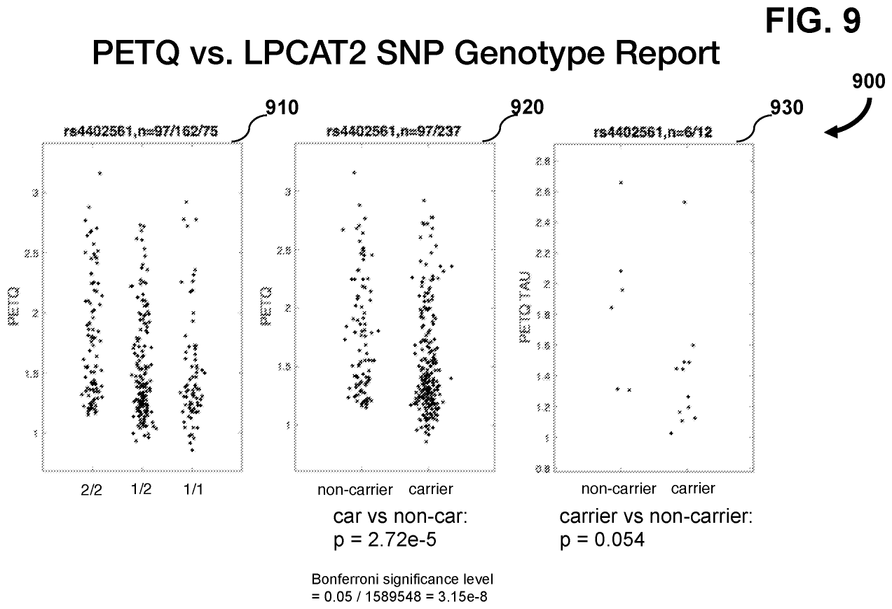

In some embodiments, the protocol can include: obtaining an accurate quantitative phenotype from image data (e.g., deep phenotype); obtaining a genotype based in part on using the accurate quantitative phenotype as a metric; determining the voxel metric; and combining the voxel metric, which can be a response tracking metric, with the accurate quantitative phenotype metric to validate the genotype, such as SNPs. For example, various aspects described with respect to FIG. 9 can be analyzed to find a SNP as a hit (e.g., p value), such as the SNPs with the lower values being indicative. Then, the carrier of the SNP can be identified as susceptible or having the condition; or being protected so as to have a reduced chance of having the condition. The carriers versus non-carriers of the SNP can then be analyzed for memory, as an indication of the status of the hippocampus.

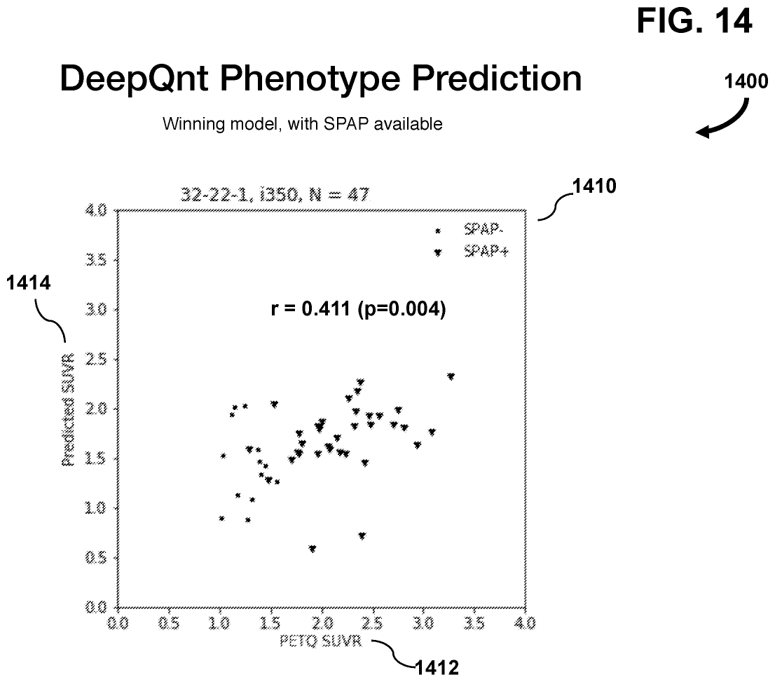

In some embodiments, deep learning can be applied in the SNP model and data may be analyzed to make the prediction of the original quantitative phenotype. This can be used as additional validation. Accordingly, the methods to obtain the SNP can be used prior to training the SNP model. The SNP model can then be used to predict the original quantitative phenotype as further validation. For example, FIG. 14 shows the predicted value vs the original quantitative phenotype, based on the SNP model.

In some embodiments, the SNP model can be tested by applying the SNP model to a dataset with amyloid positive or amyloid negative subjects. The model can also differentiate subjects with the condition versus subjects without the condition based on the SNP model. The present technology with the SNP model can be used for smaller data sets, in the order of hundreds of data points, instead of requiring large data sets with thousands of data points. This allows the SNP to be used for diagnostic purposes.

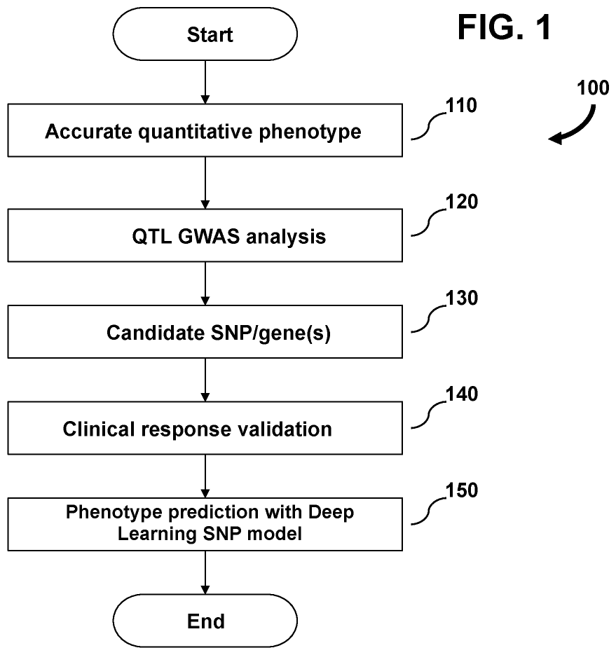

In the following description, for the purposes of explanation, specific details of the disclosure are set forth, so the disclosure can be more clearly understood. However, it will be apparent that the disclosure may be practiced by the skilled artisan without these specific details. Several specific features of example embodiments of the disclosure are detailed in the following examples, and illustrated in FIG. 1-19.

In some embodiments, FIG. 1 shows a method 100 of identifying genetic biomarkers of a condition. The method 100 can include performing a quantitative image analysis of an image of a phenotype for a condition of a subject for a plurality of subjects with the condition to obtain a first accurate quantitative phenotype. This can be referred to as obtaining an accurate quantitative phenotype as shown in Step 110. The step can be performed as described herein with respect to the disclosure and examples. In some aspects, the step can be performed by obtaining the images of the phenotype of the subjects, wherein the images accurately illustrate the condition at a given time-point. The step can also include obtaining a phenotype accuracy of greater than or about 85% for a quantitative phenotype. While the phenotype accuracy can be exactly 85%, it can be greater than 85%, or about 85% (e.g., within 1%, 2%, 3%, 4%, or 5%).

In some embodiments, the method 100 can include detecting a disease, disorder, or syndrome as the condition from the images of the phenotype of the subjects, which can be included in Step 110 or sub-step thereof. Accordingly, the images can be processed in a manner that detects a condition as a disease state, disorder, or syndrome in each subject or the one or more subjects of the plurality of subjects. Complex image analysis and database information can be used during the processing for detecting the condition. In at least one embodiment, method 100 can use multiple data points over time of a disease to perform a longitudinal analysis.

In some embodiments, the method 100 can include a process for defining a condition activity (e.g., disease activity) across a condition spectrum (e.g., disease spectrum), which can be included in Step 110 or a sub-step thereof. This can allow for identifying the stage of a condition or a progression of the condition in each subject or one or more subjects of the plurality of subjects. As such, each subject does not need to be at the same disease state in the analysis. This allows for the data time points to be a single point for each subject, but the data time points can be across different condition progression states for the population of subjects.

In some embodiments, the method 100 can include performing a standardized uptake value ratio (SUVR) analysis with the images of the phenotype of the subject. The SUVR can be implanted as known in the art. The SUVR can be computed using the image data, such as positron emission tomography (PET) images to provide a quantitative analysis of the condition as well as the state of the condition.

FIG. 1 also shows the method can include performing a quantitative genome analysis on each subject of the plurality of subjects or a plurality of different subjects as in Step 120. While the quantitative genome analysis can be performed on the same subject as in Step 110, the analysis may also be performed with a different group of subjects, which can provide a validation analysis of an independent dataset. In some aspect, the quantitative genome analysis includes performing a quantitative trait locus genome-wide association study (QTL GWAS) (e.g., Step 120). The QTL GWAS can be performed as known in the art and described herein. The result of the QTL GWAS can provide one or more SNPs, such as a panel of SNPs for analysis for correlation with the condition.

Accordingly, the result of the genome analysis can result in obtaining at least one candidate genetic biomarker for the condition in the plurality of subjects in Step 130. As such, information from the quantitative image analysis and the quantitative genome analysis can be used in Step 130 to identify the one or more candidate biomarker, which can be an SNP or gene code, such as a minor allele or major allele.

The method 100 can also include predicting a clinical response against the at least one candidate genetic biomarker to validate the at least one candidate genetic biomarker, which can be in Step 140 in order to provide the clinical response validation. This step can be useful for improving the quality of the method and can validate the candidate biomarkers that have been identified. This step can be performed with various protocols, which may specifically include Step 240 of FIG. 2 as described in more detail herein. Accordingly, the predicted clinical response can be performed to modulate at least one biological pathway associated with the at least one genetic biomarker. In some aspects, the predicted clinical response can be performed to inhibit at least one biologically active protein of a biological pathway associated with the at least one genetic biomarker. This clinical validation can be used to validate the one or more candidate biomarkers, and may be used to select one or more of the validated candidate biomarkers for further analysis and development.

In some embodiments, the predicting the clinical response can include different protocols for a clinical response validation. In some aspects, the clinical response validation can include performing a hippocampus-masked voxel-based Tau SUVR by genotype quantitation, such as described herein. In some aspect, the clinical response validation can include performing a determination of memory tracking by a voxel-based Tau SUVR by genotype quantitation with the hippocampus, such as described herein. In some aspects, the clinical response validation can include performing a voxel-based Tau quantitation within the hippocampus, such as described herein. In some aspects, the clinical response validation can include performing a Tau imaging analysis that tracks a clinical response, such as described herein. In some aspects, the clinical response validation can include performing a clinical response tracking measure. In some aspects, the clinical response validation includes at least one of: performing a tremor quantitation; or performing an automated tremor quantitation, wherein the tremor quantitation is optionally by genotype. In an example, the incorporated references can provide detailed information for tremor quantitation and the like. In some aspects, the clinical response validation can include: calculating a voxel-based serotonin receptor ligand binding from images.

The method 100 can also include performing a phenotype prediction from a genotype based on the at least one candidate genetic biomarker for the condition, which can be Step 150 for a phenotype prediction with deep learning from SNP model. In some aspects, the phenotype prediction can be used as a validation of the protocol for the identified one or more biomarkers. In some aspects, the phenotype prediction can include a quantitative phenotype analysis with positron emission tomography (PET) images. In some aspect, the phenotype prediction can predict voxel-based amyloid SUVRs with the deep learning SNP model. In some aspects, the phenotype prediction can predict voxel-based DAT-SPECT quantitation with the deep learning SNP model. In some aspects, the phenotype prediction can predict a voxel-based alpha-synuclein ligand binding with the deep learning SNP model. In some aspect, the phenotype prediction can predict regional ligand binding with the deep learning SNP model.

In some embodiments, the methods can also include an analysis of the one or more candidate biomarkers in view of one or more biological pathways associated therewith. This can include identifying at least one therapeutic target for the condition of the phenotype based on the at least one candidate genetic biomarker. The therapeutic target can be related to a candidate biomarker by either the candidate biomarker encoding for the therapeutic target or a substrate or other entity in which the therapeutic target interacts with in the one or more biological pathways associated therewith. In some aspects, the at least one therapeutic target for the condition is biologically associated with the at least one candidate genetic biomarker. Accordingly, the therapeutic targets can be selected for analysis for known or new therapeutic entities (e.g., new chemical entity (NCE)) that modulate the therapeutic target. Often, the therapeutic target can be targeted for inhibition of biological activity. In other instances, the therapeutic target can be targeted for activation and/or increasing biological activity. In an aspect, the identification of the therapeutic target can be part of Steps 120, 130, 140, or 150, or a sub-step thereof. In any event, knowing a candidate biomarker can result in identifying an associated biological pathway and analysis thereof can identify one or more therapeutic targets.

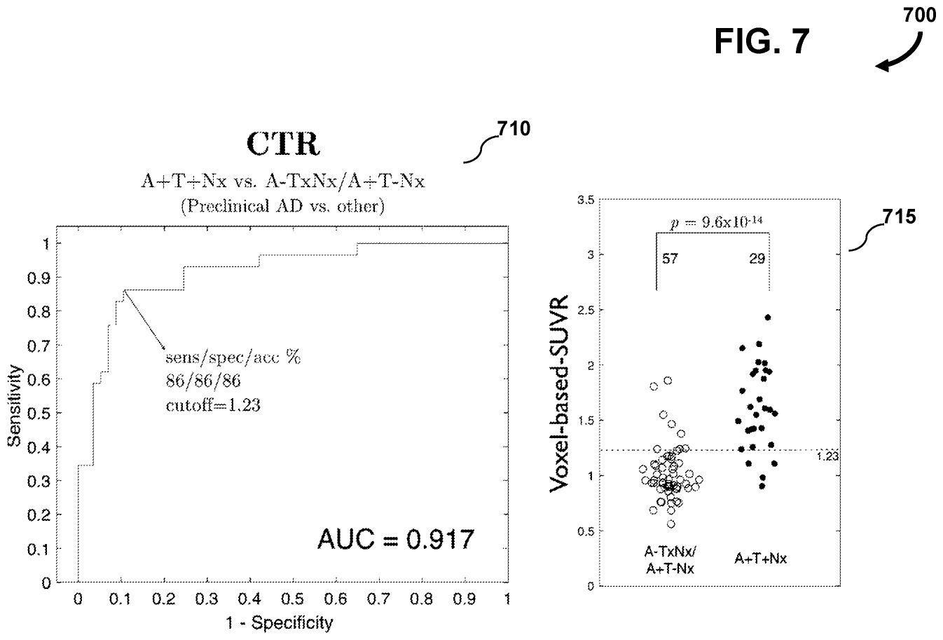

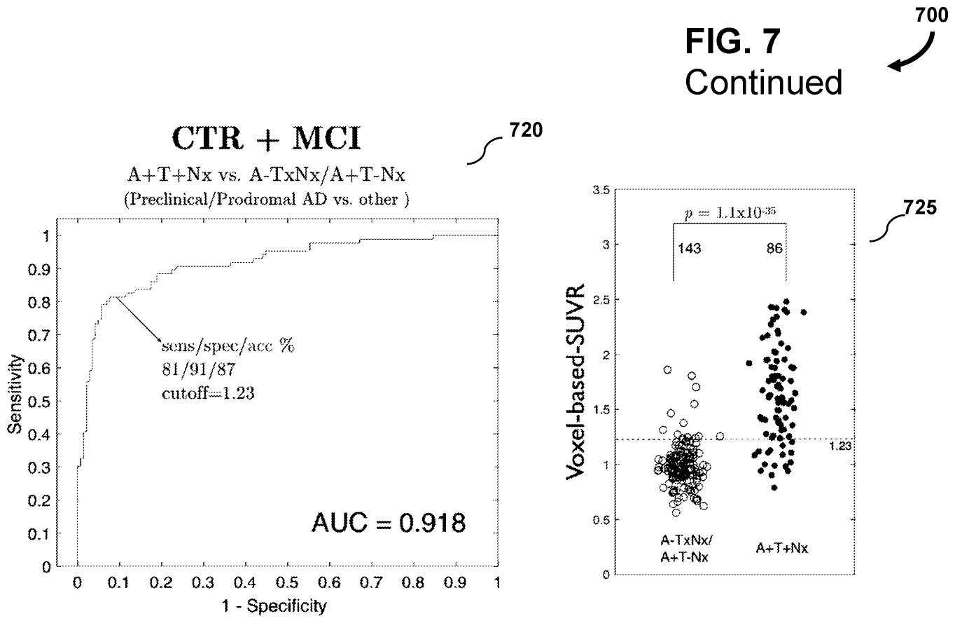

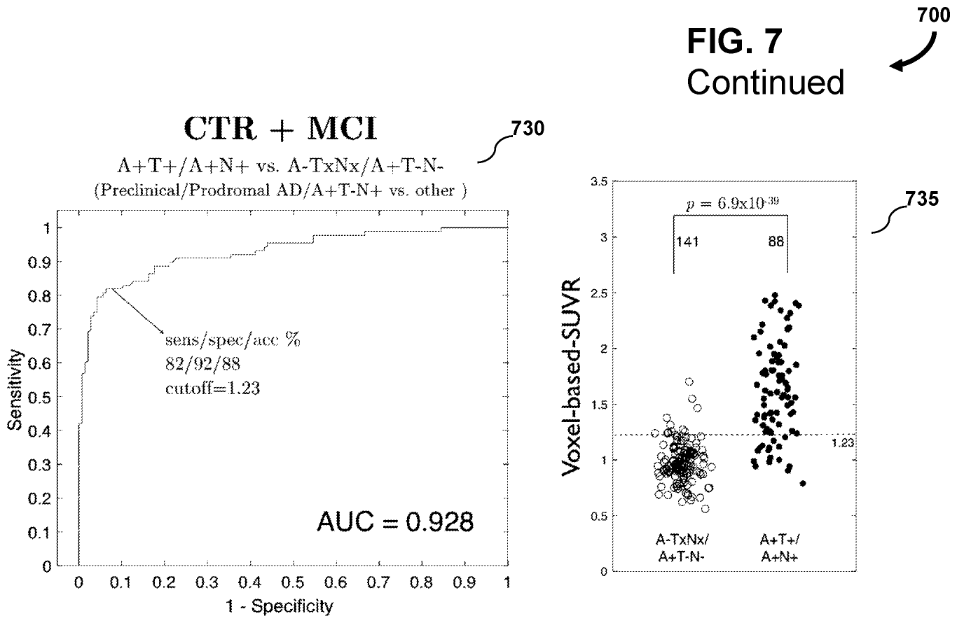

In some embodiments, the methods can also include report generation, whether paper or electronic report. The report generation can include the information obtained during the methods, such as method 100, described herein. The report can be generated and/or delivered using the protocols or systems described in the incorporated references, or may be compiled for delivery as a report for a medical practitioner. The data in the figures, such as FIGS. 7, 8, 9, 10, 11, 12, 14, 15, and/or 17-19 or similar data types or data formats may be included in the report. The report may include the resulting data, such as the determined metric. The report may also include the one or more candidate biomarkers or may include a validated and specifically identified biomarker that is associated with the condition. The report can then be provided to the one or more subjects as well as medical professionals or research scientists that work with the condition. The report may also include information about a therapeutic to be used as a therapy for the condition. As such, the methods can include generating a report with the identified least one therapeutic target for the condition. With regard to the figures, the “end” may be the end of an analysis, which can cause the report to be generated (e.g., automatically or by command by a protocol operator). In at least one embodiment, the method may recursively perform one or more of the steps of the method to refine the resulting data, such as determined metrics, candidate biomarkers, etc. In some embodiments, methods can include various actions or steps in order to perform a particular protocol. In some aspects, a method can include obtaining the images of the phenotype of the subjects, wherein the images accurately illustrate the condition at a given time-point. In some aspect, a method can include obtaining a phenotype accuracy of greater than or about or equal to 85% for a quantitative phenotype. In some aspects, a method can include detecting a disease as the condition from the images of the phenotype of the subjects. In some aspects, a method can include defining disease activity across a disease spectrum for the condition. In some aspects, the method can include performing a standardized uptake value ratio (SUVR) analysis with the images of the phenotype of the subject.

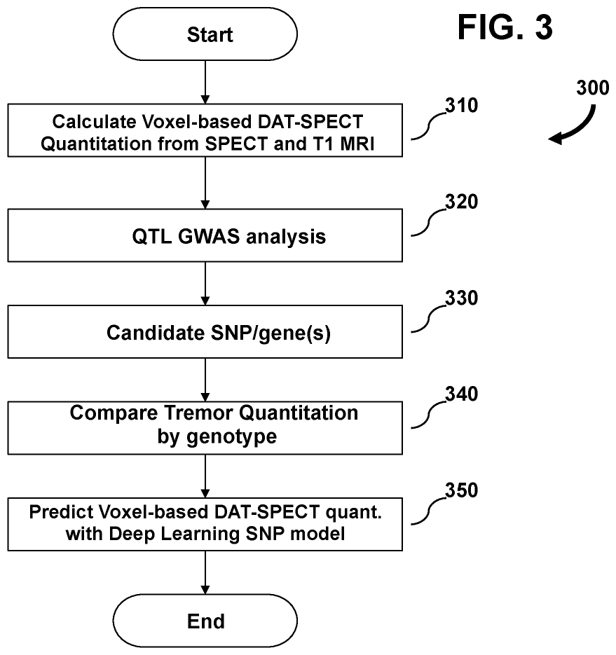

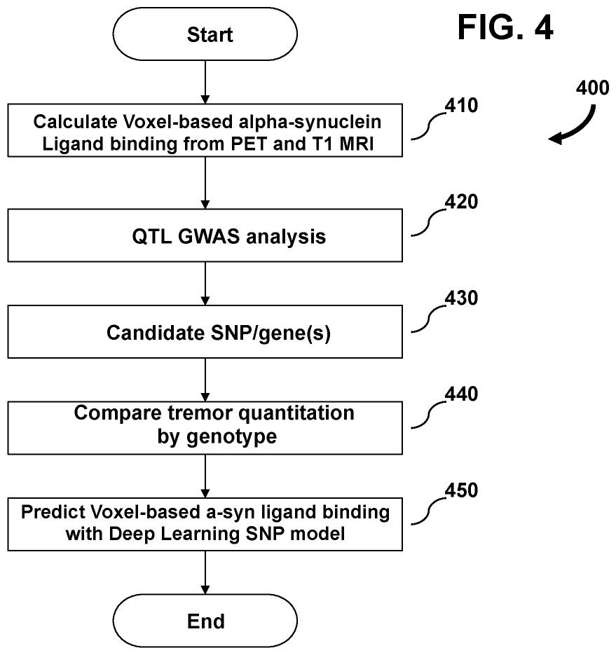

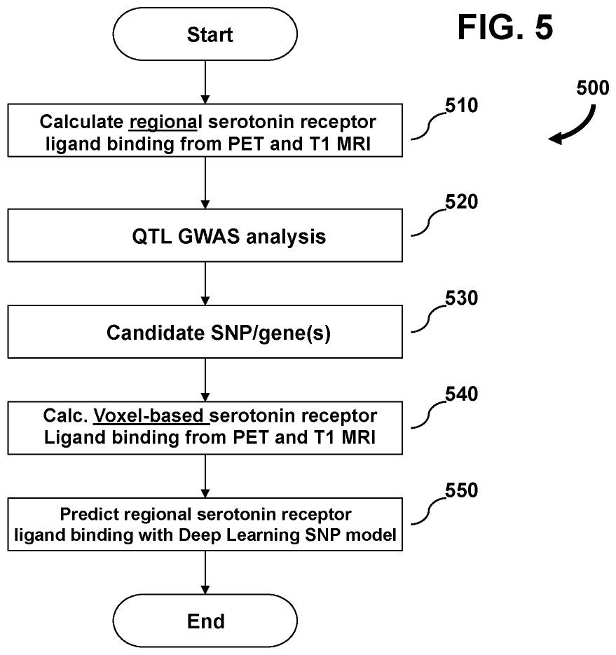

In some embodiments, the method 100 can include particular method steps for performing the quantitative image analysis of the image of the phenotype. In some aspects, the method 100 can include: calculating a voxel-based amyloid standardized uptake value ratio (SUVR) from the images (Step 210 of FIG. 2); calculating a voxel-based dopamine transporter single photon emission computed tomography (DAT-SPECT) quantitation from the images (Step 310 of FIG. 3); calculating a voxel-based alpha-synuclein ligand binding from the images (Step 410 of FIG. 4); or calculating a regional serotonin receptor ligand binding from the images (Step 510 of FIG. 5).

In some embodiments, the images used in the methods can be any image from any instrument, such as digital images from a standard camera (e.g., CCD, CMOS, etc.). However, in some aspects the images can be from imaging techniques commonly used in medical fields. These can include positron emission tomography (PET) and magnetic resonance imaging (MRI), single photon emission computed tomography (SPECT), and combinations thereof as well as others.

In some embodiments, the method 100 can include various protocols for performing the clinical response validation (Step 140). Such protocols can include at least one of: performing a hippocampus-masked voxel-based Tau SUVR by genotype quantitation; performing determination of memory tracking properties with a voxel-based Tau SUVR by genotype quantitation with the hippocampus; performing a voxel-based Tau quantitation within the hippocampus; performing a Tau imaging analysis that tracks a clinical response;

or performing a clinical response tracking measure. The clinical response validation (Step 140) may also include: comparing hippocampus-masked voxel based Tau SUVRs by genotype (Step 240 of FIG. 2); comparing tremor quantitation by genotype (Step 340 of FIG. 3 or Step 440 of FIG. 4); or calculating a voxel-based serotonin receptor ligand binding (Step 540 of FIG. 5).

As shown in FIG. 1, method 100 can include: performing an accurate quantitative phenotype analysis (Step 110); performing a quantitative trait locus genome-wide association study (QTL GWAS) (Step 120); obtaining at least one candidate genetic biomarker for the condition in the plurality of subjects from the quantitative image analysis and the quantitative genome analysis (Step 130); predicting a clinical response against the at least one candidate genetic biomarker to validate the at least one candidate genetic biomarker (Step 140); and performing a phenotype prediction with deep learning SNP model (Step 150).

FIG. 2 also provides a method 200 of identifying genetic biomarkers of a condition, which can include: calculating voxel-based amyloid SUVRs from PET and T1 MRI images (Step 210); performing a quantitative trait locus genome-wide association study (QTL GWAS) (Step 220); obtaining at least one candidate genetic biomarker for the condition in the plurality of subjects from the quantitative image analysis and the quantitative genome analysis (Step 230); comparing hippocampus-masked voxel-based Tau SUVRs by genotype (Step 240); and predicting voxel-based amyloid SUVRs with deep learning SNP model (Step 250).

FIG. 3 also provides a method 300 of identifying genetic biomarkers of a condition, which can include: calculating voxel-based DAT-SPECT quantitation from SPECT and T1 MRI images (Step 310); performing a quantitative trait locus genome-wide association study (QTL GWAS) (Step 320); obtaining at least one candidate genetic biomarker for the condition in the plurality of subjects from the quantitative image analysis and the quantitative genome analysis (Step 330); comparing tremor quantitation by genotype (Step 340); and predicting voxel-based DAT-SPECT quantitation with deep learning SNP model (Step 350).

FIG. 4 also provides a method 400 of identifying genetic biomarkers of a condition, which can include: calculating voxel-based alpha-synuclein ligand binding from PET and T1 MRI images (Step 410); performing a quantitative trait locus genome-wide association study (QTL GWAS) (Step 420); obtaining at least one candidate genetic biomarker for the condition in the plurality of subjects from the quantitative image analysis and the quantitative genome analysis (Step 430); comparing tremor quantitation by genotype (Step 440); and predicting voxel-based alpha-synuclein ligand binding with deep learning SNP model (Step 450).

FIG. 5 also provides a method 500 of identifying genetic biomarkers of a condition, which can include: calculating regional serotonin receptor ligand binding from PET and T1 MRI images (Step 510); performing a quantitative trait locus genome-wide association study (QTL GWAS) (Step 520); obtaining at least one candidate genetic biomarker for the condition in the plurality of subjects from the quantitative image analysis and the quantitative genome analysis (Step 530); calculating voxel-based serotonin receptor ligand binding with PET and T1 MRI images (Step 540); and predicting regional serotonin receptor ligand binding with deep learning SNP model (Step 550).

The embodiments illustrated and described in connection to FIGS. 1-5 are supported by the examples provided herein. The examples provide information for the protocol, data, and data analysis in order to obtain the objective to identify genetic biomarkers of a condition. The condition can be any condition, such as those listed herein. However, it should be recognized that the protocols may be applied to other conditions or subtypes thereof in order to determine other genetic biomarkers.

In some embodiments, any of the methods can include studying a biological pathway having the at least one candidate genetic biomarker that has been identified. The biological pathway may be analyzed with respect to know biological entities of the biological pathway as well as known modulators of the known biological entities. Accordingly, biological entities that can be modulated by known modulators can be identified for the therapeutic targets. The therapeutic targets may be modulated by known chemical, entities, which thereby can be included as potential therapies. These potential therapies can then be investigated for the condition. However, the known biological entities may also be studied for determining new chemical entities (NCE) that can be modulators thereof, and which may be used in therapies for the conditions. The known biological entities can be studied in silico or in real world assays, or combinations thereof.

In some embodiments, the condition is a syndrome, disease, disorder, or the like. A syndrome can have a set of medical signs and symptoms, which may be indicative of a phenotype, and that are correlated with each other in the subject(s). A disease can have a pathophysiological response to external or internal factors in the subject, which can present in the phenotype. A disorder can have a disruption to regular bodily structure and/or function in the subject.

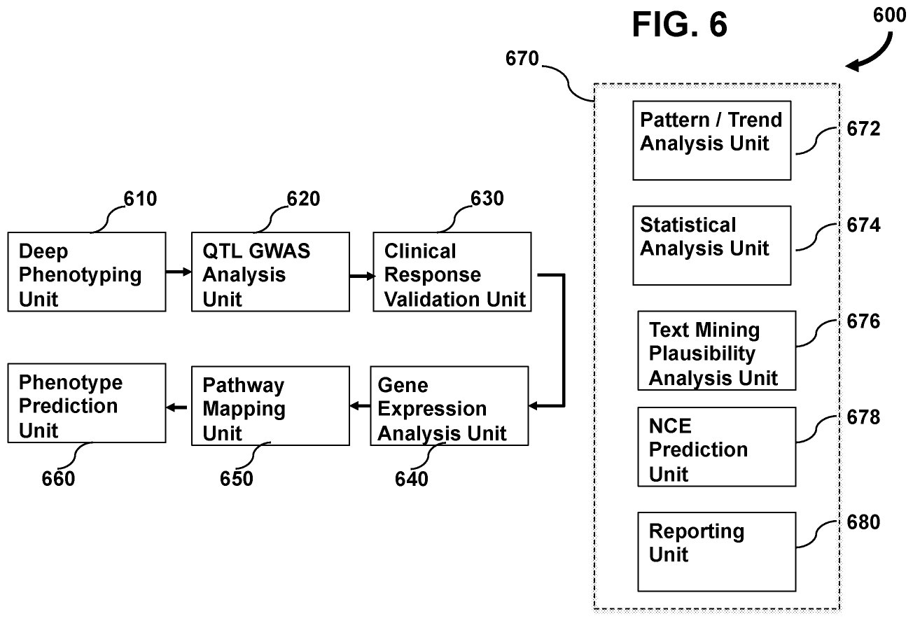

Some examples of the conditions, which are described herein, can include Alzheimer's disease, Parkinson's disease, and Major depressive disorder (MDD). However, other ailments may be used for conditions in accordance with the technology described herein. FIG. 6 shows an embodiment of a system 600 for identifying genetic biomarkers of a condition. The system 600 is shown to include at least: a deep phenotyping unit 610; a QTL GWAS analysis unit 620 downstream from the deep phenotyping unit 610; and a clinical response validation unit 630 downstream from the QTL GWAS analysis unit 620. These components can be used to perform the methods described herein. The system 600 can further (optionally) include a gene expression analysis unit 640, which can be downstream from the clinical response validation unit 630. The system 600 can further (optionally) include a pathway mapping unit 650 downstream from the clinical response validation unit 630, which can optionally also downstream from the gene expression analysis unit 640. The system 600 can further (optionally) include a phenotype prediction unit 660, which may be downstream from the clinical response validation unit 630, and/or optionally also downstream from the gene expression analysis unit 640, and/or optionally also downstream from the pathway mapping unit 650.

FIG. 6 also shows the system 600 including a toolset system 670. The toolset system 670 can include one or more units selected from: a pattern and/or trend analysis unit 672; a statistical analysis unit 674; a text mining plausibility analysis unit 676; a new chemical entity (NCE) prediction unit 678; or a reporting unit 680.

In some embodiments, the system 600 can include components that are configured in accordance with one or more of the following and in accordance with the disclosure herein. The deep phenotyping unit 610 can be configured for performing Steps 110, 210, 310, 410, and 510. The QTL GWAS analysis unit 620 can be configured for performing Steps 120, 220, 320, 420, and 520; the clinical response validation unit 630 can be configured for performing Steps 140, 240, 340, 440, and 540. The gene expression analysis unit 640 can be configured for performing gene expression profiling 640, which may or may not be automated. The pathway mapping unit 650 can be configured for mapping one or more biological pathways of genes identified by the gene expression analysis unit 640. The phenotype prediction unit 660 can be configured to use a deep learning model to predict the phenotypes identified by the deep phenotyping unit 610 or to predict new phenotypes for new investigations of new conditions. The pattern and/or trend analysis unit 672 can be used with the deep phenotyping unit 610, clinical response validation unit 630, gene expression analysis unit 640, and configured to determine patterns or trends for the phenotype. The statistical analysis unit 674 can be configured to perform statistical analyses of any data or metric of the methods. The text mining plausibility analysis unit 676 can be configured to determine therapeutic target plausibility by text mining and/or to determine a chemical modulator of a biological entity of a biological pathway associated with the therapeutic target. The new chemical entity (NCE) prediction unit 678 can be used to design or generate an NCE that can modulate the therapeutic target. The reporting unit 680 can be configured to generate reports, such as by the protocols in the incorporated references. The chemical modulator or NCE can also be substituted with a biologically active agent, which biologically active agent can be any chemical, whether biological or synthetic, such as small molecules, peptides, biologics, antibodies, nucleic acids, siRNAs, or the like.

In some embodiments, the components of the system 600 can be computers or computer modules which can be performed in computing systems. The components may include computer-executable code that when executed by a processor (e.g., microprocessor) of a computer can cause performance of the methods and method steps recited herein for any of the protocols.

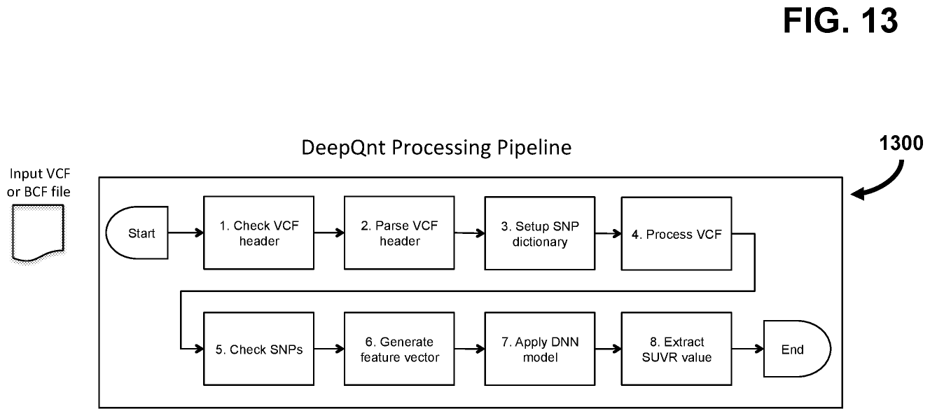

FIG. 13 shows a DeepQnt processing pipeline 1300, which can be used in the methods described herein. As shown, the processing pipeline 1300 can have the discrete steps as shown, which can be implemented with a computing system (e.g., system 600, 610, 660) and as further detailed in example 4—methods. The discrete steps can be reviewed in FIG. 13. The methods provided herein can be implemented with computing systems, such as those configured for deep learning and artificial intelligence. In some instances, the methods can include obtaining data and processing the data to obtain a recommendation for a treatment protocol. The recommended treatment protocol can then be implemented on the patient in accordance with parameters of the treatment protocol. That is, without the computational generation of the treatment protocol, the aspects of the treatment protocol cannot be performed without the instructions to do so. As such, obtaining the instructions, such as the type of drug or specific drug or combination of drugs, can be vital for performing the treatment protocol.

### EXAMPLES

### Example 1

Establishing a first integrated, quantitative “deep” Alzheimer's disease (AD) imaging phenotype 110 (FIG. 1), 210 (FIG. 2), 700 (FIG. 7) that accurately reflects disease at a given time-point (cross-sectionally) within a single measure:

Concordance Between Automated Voxel-Based Amyloid PET Quantitation and ATN Classification Now referring to FIG. 7, FIG. 7 which illustrates a diagram demonstrating high (>/=85%) accuracy (e.g., accurate phenotype) of a quantitative “deep” (e.g., metric defining disease) phenotype for target identification according to the new NIA-AA “ATN” criteria (Jack et al., Alzheimer's and Dementia 2018). The identification of the quantitative “deep” phenotype for target identification may be based on, for example, a voxel-based SUVR from an Amyloid PET and T1 MRI scan. The newly proposed “ATN” classification utilizes dichotomized biomarkers of amyloid, Tau, and neurodegeneration resulting in eight possible biomarker combinations. While “T” is motivated by AD neuropathology, it requires either a CSF sample or Tau PET. Moreover, in MCI patients “N” appears more informative regarding clinical conversion to AD (Novak et al., Polcher et al., Burnham et al., AAIC 2017). The aim of the study as demonstrated in FIG. 7 was to assess the ability of a previously (in more advanced disease) validated voxel-based amyloid PET quantitation software to identify preclinical and prodromal AD groups, as established by ATN status.

### Example 1—Methods

In an example method, 229 non-demented subjects (not used for training) were included: 86 healthy controls (CTR), 143 MCI. All subjects had complete ATN profiles available for analysis. “A”: [¹⁸F]florbetapir PET amyloid positivity was established using syngo.PET Amyloid Plaque (SPAP), a region-based quantitation method approved for clinical use. “T”: CSF p-tau status was established with the new Elecsys assay. “N”: mean left/right hippocampal volume was calculated with fast MRI quantitation previously validated against EADC/ADNI harmonized protocol. Voxel-based-SUVRs from corresponding PET/T1 MRI scan pairs were assessed for their ability to identify preclinical, or combined preclinical/prodromal groups based on ATN profiling (+, “x” denotes + or −).

Example 1—Results: Receiver operating characteristic area under the curve (AUC) graph 710 for distinguishing between preclinical AD (defined as A+T+Nx, n=29) vs. A−TxNx/A+T−Nx (n=57) CTR subjects was 0.917. In graph 710 (A+T+Nx, vs. A−TxNx/A+T−Nx (preclinical AD vs. other)), the lead line is to the sens/spec/acc %, which is 86/86/86, with a cutoff of 1.23. AUC of graph 720 for preclinical/prodromal AD (defined as A+T+Nx, n=86) vs. A−TxNx/A+T−Nx (n=143) CTR/MCI subjects was 0.918. In graph 720 (A+T+Nx vs. A-TxNx/A+T−Nx (preclinical/prodromal AD vs. other)), the lead line is to the sens/spec/acc %, which is 81/91/87, with a cutoff of 1.23. For distinguishing graph 730 between A+T+/A+N+(n=88) and A−TxNx/A+T−N− (n=141) CTR/MCI subjects AUC was 0.928. In graph 730 (A+T+/A+N+vs. A−TxNx/A+T−N− (preclinical/prodromal AD/A+T-N+vs. other)), the lead line is to the sens/spec/acc %, which is 82/92/88, with a cutoff of 1.23. In graph 715, p=9.6×10⁻¹⁴. In graph 725, p=1.1×10⁻³⁵. In graph 735, p=6.9×10⁻³⁹.

In graphs 710, 720, and 730, the X axis is 1-Specificity and the Y axis is Sensitivity. Thus, as demonstrated in FIG. 7, graph 710 voxel-based Amyloid PET quantitation showed high concordance with the ATN classification established preclinical and graph 720 preclinical/prodromal disease biomarker profiles (A+T+Nx), as determined by the reference clinical region-based quantitation method (SPAP), Elecsys assay for p-tau, and fast hippocampus volumetry. When the preclinical/MCI stage disease definition was based on A+T+ or A+N+ as previously contemplated in the NIA-AA framework (stage 2/3, MCI-AD-high) the accuracy of graph 730 was equally high.

Referring now to FIG. 6, wherein FIG. 6 is a diagram illustrating the major components as core modules 610-660, and the data flow of an example intelligent system for therapeutic target identification in drug discovery, alongside a toolset library 670 to be used with core modules 610-660; the skilled artisan will appreciate that use of a single, integrated quantitative PET measure 110 (FIG. 1), or 210 (FIG. 2) with unit 610 (FIG. 6) that accurately detects preclinical disease as defined in the new ATN classification is of key consideration as detailed in the present disclosure: in an efficient computer-implemented method 100 (FIG. 1) and system (e.g., FIG. 6) for uncovering genetic drivers of disease in neurodegeneration and other CNS diseases, from far less human data, for novel therapeutic target identification, and more specifically, in an efficient computer-implemented method 200 (FIG. 2) with system 600 (FIG. 6) for uncovering genetic drivers of disease in Alzheimer's disease, for novel therapeutic target identification.

The present disclosure describes a technique that combines and introduces a second step 140 (FIG. 1), 240 (FIG. 2) with unit 630 (FIG. 6) after obtaining said first accurate quantitate phenotype (e.g., see Step 110, Step 210, unit 610) for target identification, for example, a first voxel-based SUVR 210, 700 from an Amyloid PET and T1 MRI scan. In one embodiment, the invention then further incorporates calculation of an imaging or non-imaging quantitative measure of clinical response (e.g., see Step 140, Step 240, unit 630) against the target genotype as described further below; in one particular embodiment, said measure is a voxel-based Tau PET quantitation within the hippocampus in Step 240, wherein the quantitation in Step 240 is at least in part using a machine learning/AI-based image analysis operation to assess said clinical response in Step 140.

In the above example embodiment of method 200, the demonstrated high accuracy of the data 700 of said first imaging phenotype of Step 210 could in principle be achieved by an alternate metric of Step 110 that correlates closely with said voxel-based SUVR of Step 210 such as based on machine learning/AI.

Similarly, and in the context of the present disclosure, the memory tracking properties of said voxel-based Tau PET quantitation within the hippocampus in Step 240, could in principle be achieved by using a tight mask around the hippocampus, as already reduced to practice herein (see below example 2 details), by using said machine learning-based image analysis operation, and/or in conjunction with a selection of a specific set of voxels such as in the CA1 area of the hippocampus.

Other example embodiments of the present disclosure are outlined in FIG. 3-5:

FIG. 3 is an example of a flowchart of a computer-implemented method 300 for identifying genetic drivers of disease in Parkinson's disease, now using a voxel-based DAT-SPECT deep imaging phenotype in Step 310 calculated from DAT-SPECT and MR imaging, and automated tremor quantitation in Step 340 for clinical response assessment; alternatively or additionally, automated tremor quantitation from Step 340 could be based on machine learning/AI.

Similarly, FIG. 4 is an example of a flowchart of a computer-implemented method 400 for identifying genetic drivers of disease in Parkinson's disease, now using a voxel-based deep imaging phenotype in Step 410 calculated from alpha-synuclein PET and MR imaging, and automated tremor quantitation in Step 440 for clinical response assessment; alternatively or additionally, automated tremor quantitation from Step 440 could be based on machine learning/AI.

In yet another example, FIG. 5 is an example of a flowchart of a computer-implemented method 500 for identifying genetic drivers of disease in Major depressive disorder (MDD), using a first deep imaging phenotype in Step 510 calculated from regional serotonin receptor ligand binding from PET and T1 MRI, and a second Voxel-based serotonin receptor ligand binding in Step 540 metric from PET and T1 MRI for clinical response assessment. Alternatively or additionally, the metric (e.g., voxel metric) from Step 540 could be based on machine learning/AI.

### Example 2

Predicting clinical response by calculating hippocampus-masked voxel-based Tau SUVR: Now referring to FIG. 8, wherein FIG. 8 is a diagram demonstrating preliminary results in graph 810 and graph 820 in 30 subjects for a voxel-based Tau PET quantitation within the hippocampus 824 to assess clinical response (e.g., see Step 140, Step 240, unit 630), which showed a stronger (r=−0.60, p<0.001) correlation in graph 820 with MMSE than reported by other investigators, as it relates to either Tau PET quantitation, or Amyloid PET quantitation. In graph 810, p=0.013, with n=30, and the non-converters are the open circles with the eAD being closed circles for 12 predominantly early AD subjects (Tau scan acquired before, or within one year of conversion event in 8/12 subjects, also included 4 subjects with moderate AD). In graph 820, r=−0.60 (p<0.001) and y=−5.93x+34.2 (n=30).

The skilled artisan will appreciate that Tau PET quantitation within the hippocampus to date has been hampered in part by co-registered MRI segmentations overshooting the true borders of the hippocampus, and as a result, being polluted for example, by off-target binding in the choroid plexus; therefore, the hippocampus has been considered the wrong site for measurement because it has not been shown to correlate adequately with cognition measures such as MMSE (a gold standard for clinical response). In the present disclosure, according to an aspect of embodiment, techniques may avoid such overshooting and solve the problem herewith by incorporating a Tau imaging measure that successfully tracks clinical response.

There is considerable interest in novel approaches that accurately reflect disease activity at the earliest stage and location of neurodegeneration, and that correspond well to clinical outcome, with a particular goal of predicting and measuring response to tau-directed therapies in early Alzheimer's disease (AD). Initial attempts to derive such measures using fixed anatomical or composite regions of interest and/or z-score based approaches have so far been met with mixed success, and/or are hampered by the additional need to incorporate prior knowledge of Amyloid positivity status. Pontecorvo et al. (Brain 2017) reported promising results in a [¹⁸F]Flortaucipir PET cohort analysis. Of note, SUVR calculated from subregions of the hippocampus appear more useful for group discrimination than a composite neocortical SUVR. The aim of the study demonstrated in FIG. 8, data 800 was to demonstrate association of voxel-based (hippocampus-masked) Tau quantitation (e.g., see Step 140, Step 240, unit 630), with memory 822 (e.g., baseline MMSE), and its ability to differentiate the data in graph 810 of MCI non-converter subjects 812 from early AD subjects 814.

### Example 2—Methods

Thirty subjects 816 with MCI or AD were included (dataset not used in example 1) and divided into two groups: 18 MCI non-converters (4-5 years of MCI follow-up) and 12 predominantly early AD subjects (8/12 subjects had a PET scan acquired within one year, prior or post, the MCI-to-AD conversion event; in addition, 4/12 subjects with moderate AD were included due to limited data available at the time of abstract submission). Their corresponding [¹⁸F]Flortaucipir PET, T1 MRI scans, and MMSE scores (at the time of PET) were also obtained. A hippocampus mask was derived from the T1 MRI using a multi-atlas, GPU-accelerated, machine learning based hippocampus segmentation, as previously described and validated against the EADC/ADNI harmonized protocol (HarP). Voxel-wise SUVR maps (75-105 min, cerebellum reference) were partial volume corrected and transformed into MNI space. Optimal voxels within the hippocampus that discriminated non-converter and early AD groups were determined based on a voxel-wise t-score cluster analysis. Voxel-based-SUVRs were obtained from the hippocampus voxels for each subject in a leave-one-out analysis. Correlation between the hippocampus voxel-based-SUVR and MMSE was assessed with Pearson's correlation.

### Example 2—Results

Receiver operating characteristic area under the curve (AUC) for group separation 810 was 0.79 (two-tailed t-test for group differences: p=0.013). Hippocampus voxel-based-SUVR 824 significantly correlated with MMSE scores 822 (r=−0.60, p<0.001).

The voxel-based approach improved performance over regional hippocampal SUVR (AUC=0.60, r=−0.49) and hippocampal volume (AUC=0.73, r=0.27).

The association of [¹⁸F]Flortaucipir (18F-AV-1451) SUVR with baseline MMSE was previously demonstrated by other investigators to be on the order of ˜r=−0.4 with both global and regional measures, while the approach described herein in example 2 identified a subgroup of voxels within the hippocampus that correlates better with MMSE and in the present disclosure, according to an aspect of embodiment, solves the problem herewith by incorporating a Tau imaging measure that successfully tracks clinical response (e.g., see Step 140, Step 240, unit 630). The potential impact of choroid plexus uptake was found to be ameliorated by PVC and selection of hippocampal voxels distal to the part of the choroid plexus. Furthermore, and from a disease biology standpoint, hippocampal hyperactivity has further been investigated as a disease driver and linked to Tau deposition in prodromal AD, and therefore is of key consideration in the context of the present disclosure, in an efficient computer-implemented method 100 and system 600 for uncovering genetic drivers of disease in neurodegeneration and other CNS diseases, from far less human data, for novel therapeutic target identification, and more specifically, in an efficient computer-implemented method 200 and system 600 for uncovering genetic drivers of disease in Alzheimer's disease, from far less human data, for novel therapeutic target identification.

### Example 3

QTL GWAS Analysis against a first integrated, quantitative “deep” Alzheimer's disease (AD) imaging phenotype:

One skilled in the art may appreciate that using a high (>/=85%) accuracy-according to the new NIA-AA “ATN” criteria-quantitative “deep” phenotype (e.g., see Step 110, Step 210, unit 610) for target identification as demonstrated in example 1, in combination with a QTL GWAS analysis (e.g., Step 120, Step 220, unit 620), for example using PLINK, is a key step for identifying potentially novel SNPs. Other QTL GWAS techniques in unit 620, for example based on random forests machine learning, could also be used. High 100% accuracy of detecting more advanced disease (AD dementia) by the imaging phenotype (e.g., see Step 110, Step 210, unit 610) detailed in example 1 has previously been demonstrated, and thus said quantitative phenotype (Step 110, Step 210, unit 610) reflects disease activity across the entire disease spectrum, qualifying its use in combination with said QTL GWAS, according to the present disclosure, in an efficient computer-implemented method 200 and system 600 for uncovering genetic drivers of disease in Alzheimer's disease, from far less human data, for novel therapeutic target identification. Genotyping, in an aspect of an embodiment, was performed using the Illumina Omni2.5 microarray in a total discovery sample of n=334.

Table 1 shows a list of 33 disease associated SNPs detected as the result of said QTL GWAS 120, 220, 620 against said quantitative phenotype (e.g., see Step 110, Step 210, unit 610), according to an example of the disclosure. The respective SNP id (ID, herein not reflecting a rank), chromosome (Chr), SNP (accession number, also called rsid identifying the variant), as well as the variant alleles/bases (A1 denotes the minor allele and A2 denotes the major allele) is specified in Table 1. The minor allele can be considered the SNP for the purposes of determining the SNP showing a reduced chance or increased chance of disease. The major allele is the common allele that provides an indication opposite of what is provided by the minor allele.

TABLE 1

| ID | Chr | SNP | A1 | A2 |
|---|---|---|---|---|
| 1 | 1 | rs4912453 | T | C |
| 2 | 1 | rs12120406 | G | A |
| 3 | 1 | rs11161719 | C | T |
| 4 | 1 | rs6576798 | C | T |
| 5 | 3 | rs2030515 | G | A |
| 6 | 4 | rs4689137 | G | C |
| 7 | 4 | rs10007765 | C | G |
| 8 | 4 | rs10029820 | G | A |
| 9 | 6 | rs9458512 | A | G |
| 10 | 7 | rs1001029 | A | G |
| 11 | 7 | rs1001026 | A | G |
| 12 | 7 | rs13222318 | C | T |
| 13 | 7 | rs62444137 | A | G |
| 14 | 7 | rs17680408 | A | G |
| 15 | 7 | rs10234008 | T | C |
| 16 | 7 | rs917321 | T | C |
| 17 | 7 | rs993900 | A | G |
| 18 | 14 | rs8009420 | A | C |
| 19 | 14 | rs213563 | A | G |
| 20 | 15 | rs7164265 | C | T |
| 21 | 15 | rs1551466 | C | G |
| 22 | 15 | rs12916234 | A | C |
| 23 | 15 | rs9920618 | C | T |
| 24 | 16 | rs8056050 | C | T |
| 25 | 18 | rs4891826 | G | T |
| 26 | 19 | rs71352238 | C | T |
| 27 | 19 | rs2075650 | G | A |
| 28 | 19 | rs157582 | A | G |
| 29 | 19 | rs769449 | A | G |
| 30 | 19 | rs56131196 | A | G |
| 31 | 19 | rs4420638 | G | A |
| 32 | 19 | rs17815373 | A | G |
| 33 | 16 | rs4402561 | C | T |

The above detailed steps 110-150, 210-250 and units 610, 620, 630 in the intelligent system 600 and computer-implemented methods 100 and 200 disclosed herein may further be followed by automated steps to execute gene expression profiling in unit 640, and target plausibility analysis in units 672, 674, 676, 680 including pathway mapping unit 650. A set of candidate SNPs/genes such as listed in Table 1, according to one embodiment, may further be constructed to accurately predict said first imaging phenotype (e.g., see Step 110, Step 210, unit 610) with a deep learning model (e.g., see Step 150, Step 250, unit 660) from said SNPs/genes (e.g., see Step 130, Step 230), to serve as additional validation step.

### Example 4

Phenotype prediction from far less genotype data with a deep learning based system (DeepQnt) within the intelligent system for therapeutic target identification in drug discovery, and head-to-head performance comparison with a polygenic hazard score (PHS, Desikan et al. PLoS Medicine 2017) derived from thousands of subjects:

Referring to FIG. 14, FIG. 14 illustrates a diagram demonstrating phenotype (PETQ SUVR) (e.g., see Step 110, Step 210, unit 610), which can provide the PETQ SUVR 1412 prediction from genotype with a deep learning based system (DeepQnt) (e.g., see Step 150, Step 250, unit 660) within the disclosed computer-implemented method (e.g., method 100 or method 200) implemented with intelligent system 600 for therapeutic target identification in drug discovery; subjects that have an Amyloid positive PET scan (assessed by an FDA approved method, SPAP) are denoted SPAP+ (triangle symbol). The prediction 1414 (e.g., predicted SUVR) may achieve a significant correlation with a Pearson's r=0.411 against the original quantitative phenotype obtained by, for example Step 110 or Step 210 with unit 610 to obtain the PETQ SUVR 1412.

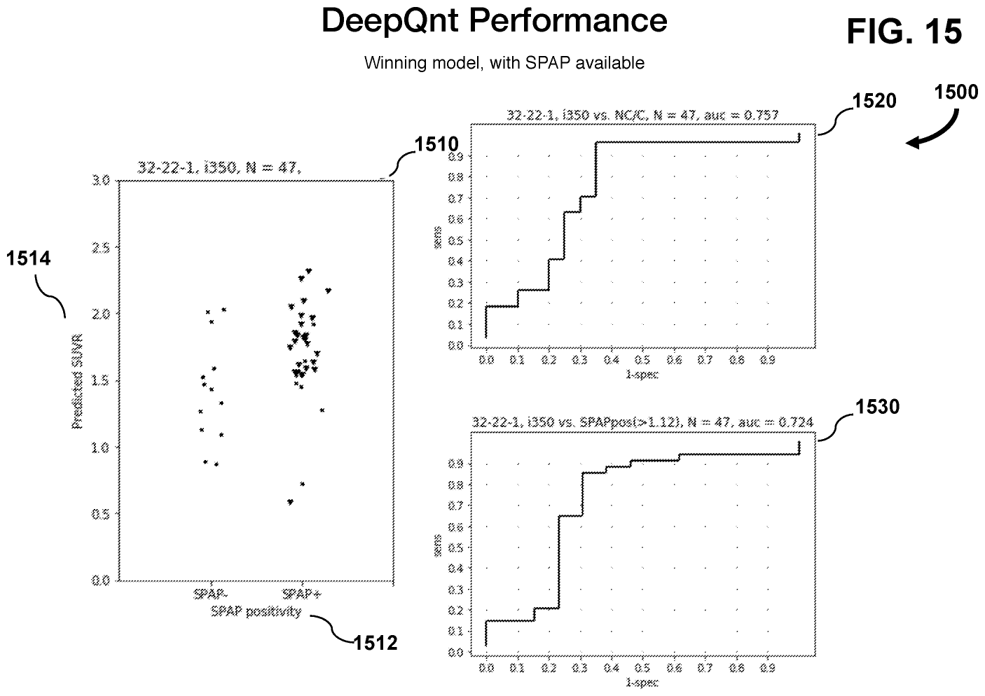

Referring to FIG. 15, wherein FIG. 15 is a diagram demonstrating the performance of an example phenotype predictor (DeepQnt) with, for example, Step 150 or Step 250 with unit 660 within the disclosed computer-implemented method 100 or method 200 and with the intelligent system 600 for therapeutic target identification in drug discovery; SPAPpos denotes a Amyloid positive PET scan, triangle symbol denotes converters to AD from MCI, NC/C conversion to AD from mild cognitive impairment (MCI) in a dataset with long (>/=5 years) follow-up.

In graph 1510, the prediction 1514 (e.g., predicted SUVR) allows not only to separate the groups by Amyloid positivity status 1512 (e.g., SPAP positivity), but also to separate converters C (triangle symbol) from non-converters NC (square symbol). The respective Receiver Operating Characteristic (ROC) curves are shown in graph 1520 (Area under the curve, AUC=0.757 for C vs NC) and graph 1530 (AUC 0.724 for Amyloid positivity detection).

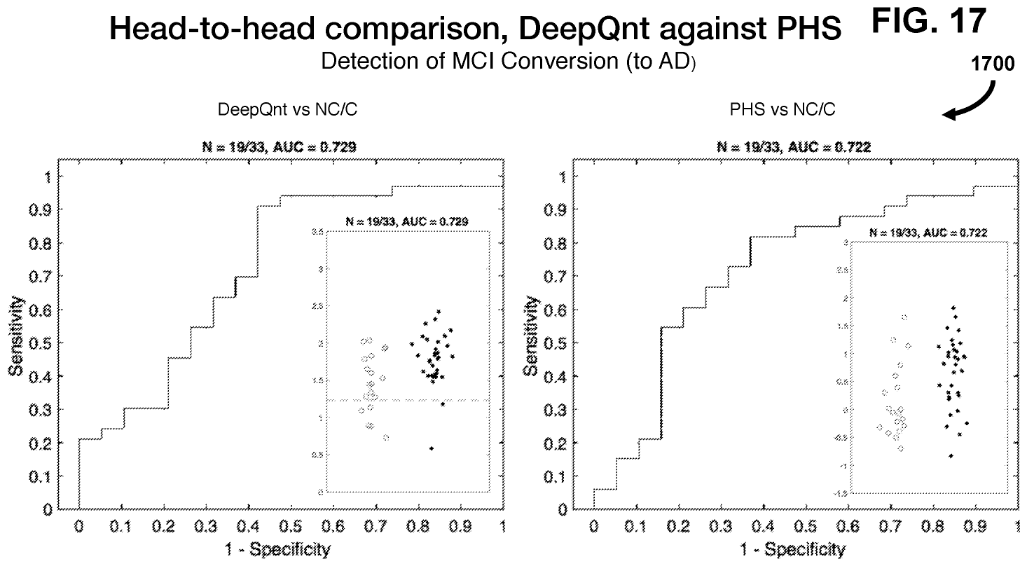

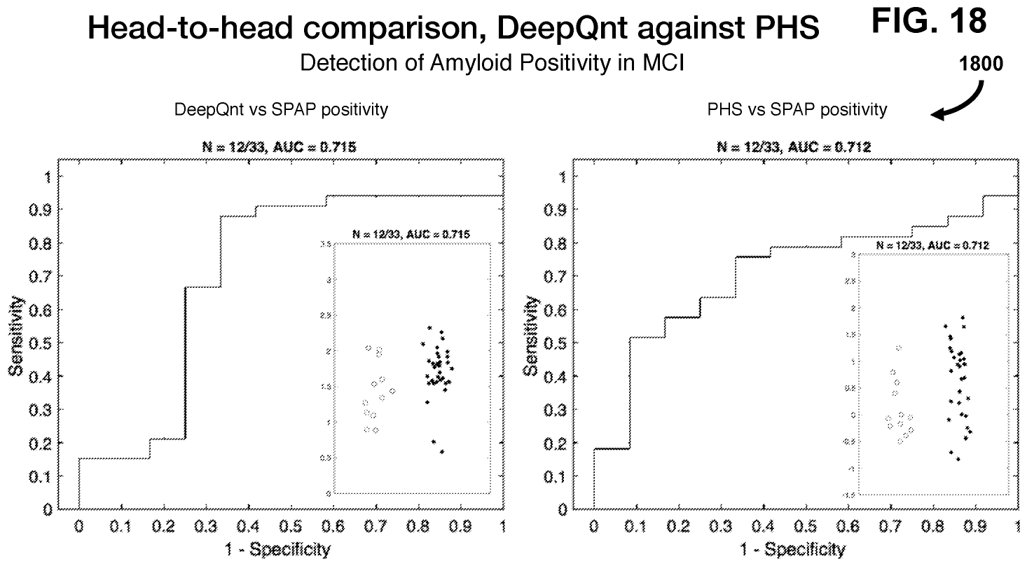

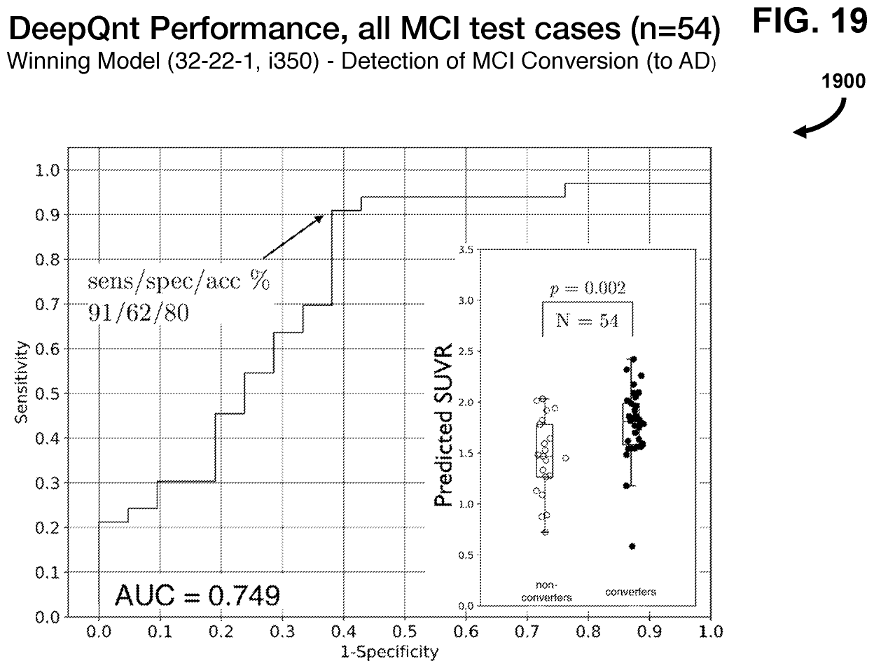

These performance results, by way of example, demonstrate that the powerful computer technique in Alzheimer's Disease (AD) allows to discover therapeutic targets with just a few hundred samples, and achieve comparable performance on par with other gene discovery/risk prediction approaches such as a polygenic hazard score (PHS) derived from thousands of subjects (detailed data shown in FIGS. 17-18) and improved performance for detecting early AD (detection of MCI conversion to AD; sensitivity 91%, specificity 62%, accuracy 80%; FIG. 19) compared to APOE4 carrier status alone (sensitivity 76%, specificity 67%, accuracy 72%, data not shown).

### Example 4—Methods

For training, a total of 334 subjects were included: 128 healthy controls (CTR), 159 MCI, and 47 AD. For testing, a separate dataset of 54 MCI subjects were included and divided into converters (to AD) and non-converters: 33 converters (PET 1 to 3 years prior to conversion) and 21 non-converters (5-7 years of MCI follow-up). Their corresponding [¹⁸F]florbetapir PET, T1 MRI, Illumina Omni2.5M genotyping data, reference clinical region-based quantitation method (SPAP) and used for establishing Amyloid positivity (SPAP+) status, and polygenic hazard score (PHS), were obtained; only a subset of the 54 MCI cases (52) had also PHS, and 47 had also SPAP available; 45 cases had both PHS and SPAP. A first voxel-based-SUVR imaging phenotype (e.g., Step 110, Step 210, unit 610) to obtain the PETQ SUVR 1412, was calculated for each PET/MRI scan pair, as detailed in example 1.

An additional dataset (not used for training) with a second clinical response measure from a voxel-based Tau PET quantitation within the hippocampus (e.g., see Step 140, Step 240, unit 630), wherein the quantitation is at least in part using a machine learning/AI-based image analysis operation, was further used to validate said clinical response, as detailed in example 2.

PLINK v1.9 beta (linear dominant model, MAF>0.1) (e.g., see Step 120, Step 220, unit 620) was used to detect candidate SNPs (e.g., Step 130, Step 230) associated with the voxel-based-SUVR phenotype (e.g., see Step 110, Step 210, unit 610, PETQ SUVR 1412), using a model without covariates and a model with APOE4 carrier status as a covariate. An artificial neural network (ANN) was trained using a set of 32 SNPs (see Table 1, SNP id 1-32) to predict the first voxel-based-SUVR phenotype (e.g., Step 110, Step 210, unit 610, PETQ SUVR 1412); using GPU-accelerated TensorFlow (v1.1.0) and selected parameters (batch size=4, keep probability=1.0, initializer=Xavier, optimizer=Adam, learning rate=0.001, layers=[32-22-1], iterations=350). A predicted SUVR 1414, 1514 was calculated for each test subject with the ANN model. One skilled in the art will appreciate that in some aspects, the ANN may further be trained using genotype data comprising only a subset of said SNP id 1-32 in Table 1, and optionally SNP id 33 of Table 1 herein to predict said first voxel-based-SUVR phenotype. The ANN may, for example, also be implemented using toolkits such Caffe, Pytorch; or MATLAB, and then compiled as executable or C/C++ shared library.

Referring to FIG. 13, FIG. 13 illustrates a diagram illustrating the major components, and the data flow/processing pipeline (step 1-8) in an example phenotype predictor (DeepQnt) (e.g., see Step 150, Step 250, unit 660) within the intelligent system 600 for therapeutic target identification in drug discovery; a set of candidate SNPs/genes (e.g., see Step 130, Step 230) may be constructed to predict the original imaging phenotype (e.g., see Step 110, Step 210, unit 610, PETQ SUVR 1412) with a deep learning model from discovered SNPs/genes and may serve as an additional validation step.

In greater detail, pseudocode for the discrete steps in FIG. 13 is as following, in one embodiment:
1. Read input file (VCF/BCF) header, check samples, check for tabix file, and update settings;
2. Extract the raw header, number of samples, sequence names, ##reference, and ##fileformat;
3. Create dictionary of key/value associations for each SNP, including genotype, ploidy, #occurrences;
4. Read each row of the input file and:
   a. Compare against the SNP dictionary keys to check for a match;
   b. Determine the type of match made (rs+bp, rs only, bp only);
   c. Extract REF letter, ALT letter, genotype and letter codes;
   d. Based on the genotype, assign the appropriate tensorflow model code (1, 2, or 3);
   e. Add the extracted information and model codes to the SNP dictionary; 5. Print SNP info to log file and check whether all required SNPs are present;
6. Generate feature vector from the SNP genotype data for input to tensorflow model;
7. Recode genotypes to non-carrier/carrier and run tensorflow DNN model; and
8. Convert the output from tensorflow model into a formatted SUVR output value, print to terminal.

### Example 5

Discovery of Alzheimer's Disease LPCAT2 candidate gene and associated SNP rs4402561 with the disclosure.

Referring now to FIG. 9, FIG. 9 is an example of a report generated by a system 600, such as with unit 680, for uncovering genetic drivers of disease in Alzheimer's disease, here by way of example the candidate LPCAT2 gene/protein; as the skilled artisan will appreciate, a suggestive association of graph 920 for protective candidate SNP (e.g., see Step 130, Step 230) rs4402561 (see Table 1, SNP id 33) has been detected with a first accurate quantitative phenotype (PETQ) (e.g., Step 110, Step 210, unit 610, PETQ SUVR 1412) and the step-down pattern in graph 930, (e.g., see unit 672, unit 674) confirmed (trailing p=0.054) by a second clinical response tracking measure (PETQ TAU) (e.g. Step 140, Step 240, unit 630). Graph 910 shows data for rs4402561 with n=97/162/75 (left data is 2/2; middle data is 1/2; right data is 1/1). Graph 920 shows data for rs4402561 with n=97/237 (left data is non-carrier and right data is carrier), with p=2.72e-5. The Bonferroni significance level=0.05/1589548=3.15e-8. Graph 930 shows data for rs4402561 with n=6/12 (left data is non-carrier and right data is carrier), with p=0.054.

In Table 1, id 33, the base corresponding to the protective property of the rs4402561 SNP (e.g., Step 130, Step 230) as revealed by the QTL GWAS (e.g., Step 120, Step 220, unit 620) can be found in column A1 for the minor allele. One skilled in the art will appreciate that even though A1 is the minor allele in this QTL GWAS example, the designation of minor allele (MA) is depending on the population minor allele frequency (MAF) used for the association study, and actual base letter substitution may need to be translated depending on genotyping method and coding/reporting standard used during analysis. For example, for rs4402561 the base revealed as protection associated is C, however if reported in reverse strand orientation it is G.

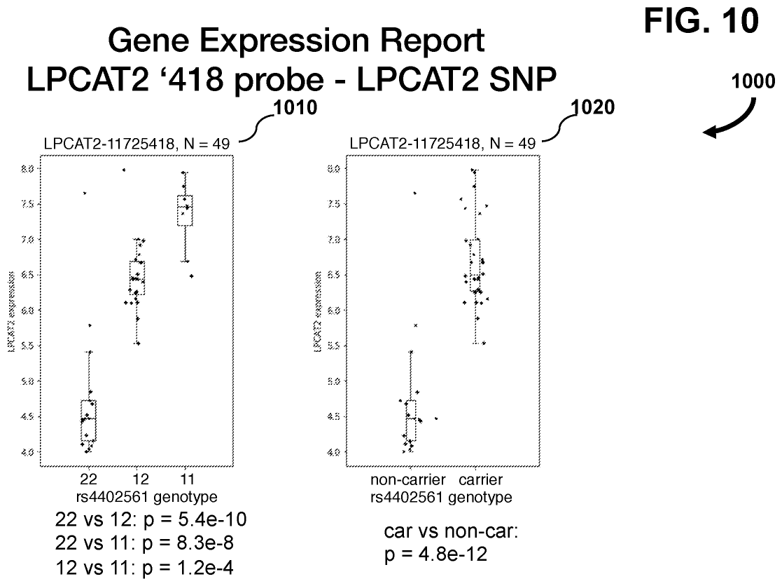

Referring to FIG. 10, FIG. 10 illustrates an example of a report 1000 generated by a system 600, 680 for uncovering genetic drivers of disease in Alzheimer's disease, here by way of example the candidate LPCAT2 gene/protein; as one skilled in the art will appreciate, blood expression profiling graphs 1010, 1020, confirms a highly significant step-up pattern 1010, 672, 674 of a LPCAT2 probe possibly associated with a regulatory transcript. Graphs 1010 and 1020 are for LPCAT2−11725418, N=49. In Graph 1010, the left data is 22, middle data is 12, and right data is 11. In Graph 1020, the left data is non-carrier, and the right data is carrier.

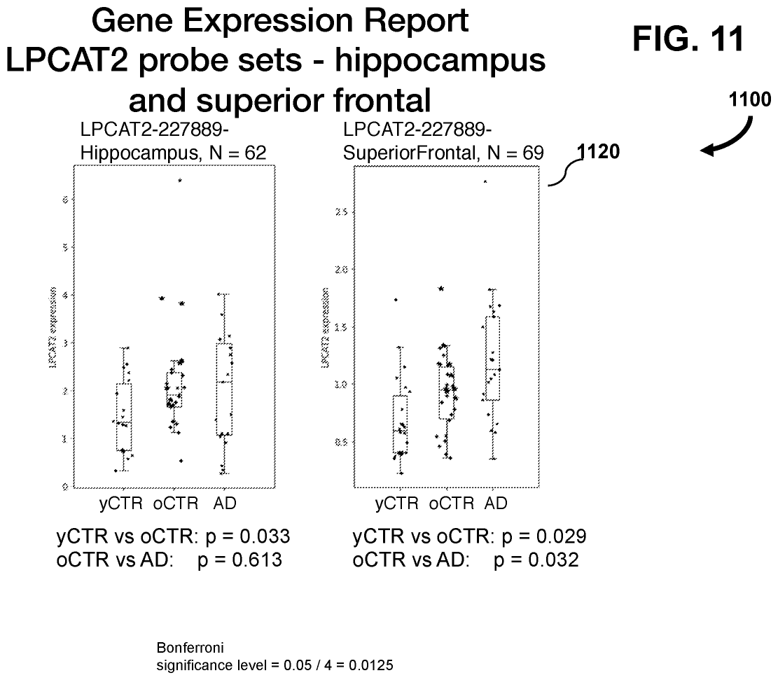

Referring to FIG. 11, FIG. 11 illustrates an example of a report 1100 generated by a system 600, such as with unit 680, for uncovering genetic drivers of disease in Alzheimer's disease, here by way of example the candidate LPCAT2 gene/protein; the skilled artisan will appreciate that brain bank gene expression profiling 1120 confirms a trending step-up pattern shown in profile 1120 (e.g., see unit 672, unit 674) of a LPCAT2 probe between young controls (yCTR), age-matched older controls (oCTR) and Alzheimer's disease subjects (AD), within the superior frontal cortex. The data in the left graph is for LPCAT-227889—Hippocampus, N=62 (left data is yCTR, middle data is oCTR, and right data is AD), and the data in the right graph is for LPCAT2−227889—Superior frontal, N=69 (left data is yCTR, middle data is oCTR, and right data is AD). The Bonferroni significance level=0.05/4=0.0125.

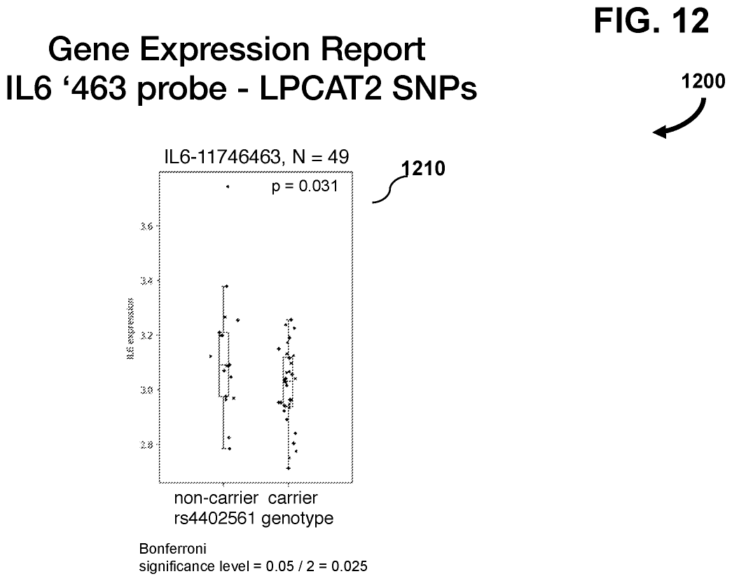

Referring now to FIG. 12, FIG. 12 illustrates an example of a report 1200 generated by a system 600, such as with unit 680, for uncovering genetic drivers of disease in Alzheimer's disease, here by way of example for the candidate LPCAT2 gene/protein; one skilled in the art will appreciate that blood expression profiling 1210 of pro-inflammatory cytokine IL6 by rs4402561 minor allele carrier status confirms a trending step-down pattern shown in the profile 1210 (e.g., see unit 672, unit 674) and explaining protection. The data is for IL6-11746463, N=49, and p=0.031 (left data is non-carrier; right data is carrier). The Bonferroni significance level=0.05/2=0.025.

In the above example 5, discovery of Alzheimer's Disease LPCAT2 candidate gene and associated SNP rs4402561, the putative therapeutic target is further substantiated by up/downstream pathway mapping with unit 650 and target plausibility analysis by text mining with unit 676. The downstream molecular pathway for LPCAT leads to Platelet Activation Factor (PAF), which is further expressed in microglia cells. A small-molecule selective LPCAT2 inhibitor (TSI-01) has been found with unit 676, the respective SMILES code thereof being:

O═C(C(C1)=C1C1)N(C2=CC═C(C(OC(C)C)═O)C=C2)C1=O.

Starting with a known molecular structure allows for structure based drug design with unit 678, using tools such as SwissTargetPrediction. Lastly, the LPCAT2 putative target by itself could further be used to discover possible new chemical entities (NCEs) in-silico, for example using Atomwise AtomNet, or other AI-based in-silico drug discovery tools of unit 678.

The present disclosure is not to be limited in terms of the particular embodiments described in this application, which are intended as illustrations of various aspects. Many modifications and variations can be made without departing from its spirit and scope. Functionally equivalent methods and apparatuses within the scope of the disclosure, in addition to those enumerated herein, are possible from the foregoing descriptions. The terminology used herein is for the purpose of describing particular embodiments only, and is not intended to be limiting.

In one embodiment, the present methods can include aspects performed on a computing system. As such, the computing system can include a memory device that has the computer-executable instructions for performing the methods. The computer-executable instructions can be part of a computer program product that includes one or more algorithms for performing any of the methods described herein.

In one embodiment, any of the operations, processes, or methods, described herein can be performed or cause to be performed in response to execution of computer-readable instructions stored on a computer-readable medium and executable by one or more processors. The computer-readable instructions can be executed by a processor of a wide range of computing systems from desktop computing systems, portable computing systems, tablet computing systems, hand-held computing systems, as well as network elements, and/or any other computing device. The computer readable medium is not transitory. The computer readable medium is a physical medium having the computer-readable instructions stored therein so as to be physically readable from the physical medium by the computer/processor.

There are various vehicles by which processes and/or systems and/or other technologies described herein can be effected (e.g., hardware, software, and/or firmware), and that the preferred vehicle may vary with the context in which the processes and/or systems and/or other technologies are deployed. For example, if an implementer determines that speed and accuracy are paramount, the implementer may opt for a mainly hardware and/or firmware vehicle; if flexibility is paramount, the implementer may opt for a mainly software implementation; or, yet again alternatively, the implementer may opt for some combination of hardware, software, and/or firmware.

The various operations described herein can be implemented, individually and/or collectively, by a wide range of hardware, software, firmware, or virtually any combination thereof. In one embodiment, several portions of the subject matter described herein may be implemented via application specific integrated circuits (ASICs), field programmable gate arrays (FPGAs), digital signal processors (DSPs), or other integrated formats. However, some aspects of the embodiments disclosed herein, in whole or in part, can be equivalently implemented in integrated circuits, as one or more computer programs running on one or more computers (e.g., as one or more programs running on one or more computer systems), as one or more programs running on one or more processors (e.g., as one or more programs running on one or more microprocessors), as firmware, or as virtually any combination thereof, and that designing the circuitry and/or writing the code for the software and/or firmware are possible in light of this disclosure. In addition, the mechanisms of the subject matter described herein are capable of being distributed as a program product in a variety of forms, and that an illustrative embodiment of the subject matter described herein applies regardless of the particular type of signal bearing medium used to actually carry out the distribution. Examples of a physical signal bearing medium include, but are not limited to, the following: a recordable type medium such as a floppy disk, a hard disk drive (HDD), a compact disc (CD), a digital versatile disc (DVD), a digital tape, a computer memory, or any other physical medium that is not transitory or a transmission. Examples of physical media having computer-readable instructions omit transitory or transmission type media such as a digital and/or an analog communication medium (e.g., a fiber optic cable, a waveguide, a wired communication link, a wireless communication link, etc.).

It is common to describe devices and/or processes in the fashion set forth herein, and thereafter use engineering practices to integrate such described devices and/or processes into data processing systems. That is, at least a portion of the devices and/or processes described herein can be integrated into a data processing system via a reasonable amount of experimentation. A typical data processing system generally includes one or more of a system unit housing, a video display device, a memory such as volatile and non-volatile memory, processors such as microprocessors and digital signal processors, computational entities such as operating systems, drivers, graphical user interfaces, and applications programs, one or more interaction devices, such as a touch pad or screen, and/or control systems, including feedback loops and control motors (e.g., feedback for sensing position and/or velocity; control motors for moving and/or adjusting components and/or quantities). A typical data processing system may be implemented utilizing any suitable commercially available components, such as those generally found in data computing/communication and/or network computing/communication systems.

The herein described subject matter sometimes illustrates different components contained within, or connected with, different other components. Such depicted architectures are merely exemplary, and that in fact, many other architectures can be implemented which achieve the same functionality. In a conceptual sense, any arrangement of components to achieve the same functionality is effectively “associated” such that the desired functionality is achieved. Hence, any two components herein combined to achieve a particular functionality can be seen as “associated with” each other such that the desired functionality is achieved, irrespective of architectures or intermedial components. Likewise, any two components so associated can also be viewed as being “operably connected”, or “operably coupled”, to each other to achieve the desired functionality, and any two components capable of being so associated can also be viewed as being “operably couplable”, to each other to achieve the desired functionality. Specific examples of operably couplable include, but are not limited to: physically mateable and/or physically interacting components and/or wirelessly interactable and/or wirelessly interacting components and/or logically interacting and/or logically interactable components.

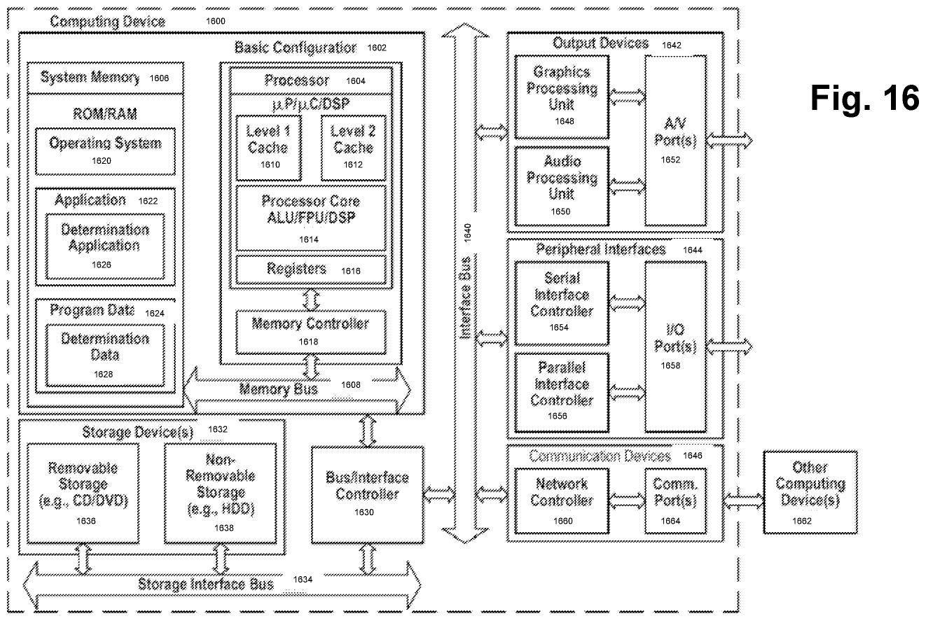

FIG. 16 shows an example computing device 1600 (e.g., a computer) that may be arranged in some embodiments to perform the methods (or portions thereof) described herein. In a very basic configuration 1602, computing device 1600 generally includes one or more processors 1604 and a system memory 1606. A memory bus 1608 may be used for communicating between processor 1604 and system memory 1606.

Depending on the desired configuration, processor 1604 may be of any type including, but not limited to: a microprocessor (μP), a microcontroller (μC), a digital signal processor (DSP), or any combination thereof. Processor 1604 may include one or more levels of caching, such as a level one cache 1610 and a level two cache 1612, a processor core 1614, and registers 1616. An example processor core 1614 may include an arithmetic logic unit (ALU), a floating point unit (FPU), a digital signal processing core (DSP Core), or any combination thereof. An example memory controller 1618 may also be used with processor 1604, or in some implementations, memory controller 1618 may be an internal part of processor 1604.

Depending on the desired configuration, system memory 1606 may be of any type including, but not limited to: volatile memory (such as RAM), non-volatile memory (such as ROM, flash memory, etc.), or any combination thereof. System memory 1606 may include an operating system 1620, one or more applications 1622, and program data 1624. Application 1622 may include a determination application 1626 that is arranged to perform the operations as described herein, including those described with respect to methods described herein. The determination application 1626 can obtain data, such as pressure, flow rate, and/or temperature, and then determine a change to the system to change the pressure, flow rate, and/or temperature.

Computing device 1600 may have additional features or functionality, and additional interfaces to facilitate communications between basic configuration 1602 and any required devices and interfaces. For example, a bus/interface controller 1630 may be used to facilitate communications between basic configuration 1602 and one or more data storage devices 1632 via a storage interface bus 1634. Data storage devices 1632 may be removable storage devices 1636, non-removable storage devices 1638, or a combination thereof. Examples of removable storage and non-removable storage devices include: magnetic disk devices such as flexible disk drives and hard-disk drives (HDD), optical disk drives such as compact disk (CD) drives or digital versatile disk (DVD) drives, solid state drives (SSD), and tape drives to name a few. Example computer storage media may include: volatile and non-volatile, removable and non-removable media implemented in any method or technology for storage of information, such as computer readable instructions, data structures, program modules, or other data.

System memory 1606, removable storage devices 1636 and non-removable storage devices 1638 are examples of computer storage media. Computer storage media includes, but is not limited to: RAM, ROM, EEPROM, flash memory or other memory technology, CD-ROM, digital versatile disks (DVD) or other optical storage, magnetic cassettes, magnetic tape, magnetic disk storage or other magnetic storage devices, or any other medium which may be used to store the desired information and which may be accessed by computing device 1600. Any such computer storage media may be part of computing device 1600. Computing device 1600 may also include an interface bus 1640 for facilitating communication from various interface devices (e.g., output devices 1642, peripheral interfaces 1644, and communication devices 1646) to basic configuration 1602 via bus/interface controller 1630. Example output devices 1642 include a graphics processing unit 1648 and an audio processing unit 1650, which may be configured to communicate to various external devices such as a display or speakers via one or more A/V ports 1652. Example peripheral interfaces 1644 include a serial interface controller 1654 or a parallel interface controller 1656, which may be configured to communicate with external devices such as input devices (e.g., keyboard, mouse, pen, voice input device, touch input device, etc.) or other peripheral devices (e.g., printer, scanner, etc.) via one or more I/O ports 1658. An example communication device 1646 includes a network controller 1660, which may be arranged to facilitate communications with one or more other computing devices 1662 over a network communication link via one or more communication ports 1664.

The network communication link may be one example of a communication media. Communication media may generally be embodied by computer readable instructions, data structures, program modules, or other data in a modulated data signal, such as a carrier wave or other transport mechanism, and may include any information delivery media. A “modulated data signal” may be a signal that has one or more of its characteristics set or changed in such a manner as to encode information in the signal. By way of example, and not limitation, communication media may include wired media such as a wired network or direct-wired connection, and wireless media such as acoustic, radio frequency (RF), microwave, infrared (IR), and other wireless media. The term computer readable media as used herein may include both storage media and communication media.

Computing device 1600 may be implemented as a portion of a small-form factor portable (or mobile) electronic device such as a cell phone, a personal data assistant (PDA), a personal media player device, a wireless web-watch device, a personal headset device, an application specific device, or a hybrid device that includes any of the above functions. Computing device 1600 may also be implemented as a personal computer including both laptop computer and non-laptop computer configurations. The computing device 1600 can also be any type of network computing device. The computing device 1600 can also be an automated system as described herein.

The embodiments described herein may include the use of a special purpose computer including various computer hardware or software modules.

Embodiments within the scope of the present disclosure also include computer-readable media for carrying or having computer-executable instructions or data structures stored thereon. Such computer-readable media can be any available media that can be accessed by a computer. By way of example, and not limitation, such computer-readable media can comprise RAM, ROM, EEPROM, CD-ROM or other optical disk storage, magnetic disk storage or other magnetic storage devices, or any other medium which can be used to carry or store desired program code means in the form of computer-executable instructions or data structures and which can be accessed by a computer. When information is transferred or provided over a network or another communications connection (either hardwired, wireless, or a combination of hardwired or wireless) to a computer, the computer properly views the connection as a computer-readable medium. Thus, any such connection is properly termed a computer-readable medium. Combinations of the above should also be included within the scope of computer-readable media.

Computer-executable instructions comprise, for example, instructions and data which cause a computer, or special purpose processing device to perform a certain function or group of functions. Although the subject matter has been described in language specific to structural features and/or methodological acts, it is to be understood that the subject matter described herein is not limited to the specific features or acts described above.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number is intended, such an intent will be explicitly recited, and in the absence of such recitation, no such intent is present. For example, as an aid to understanding, the description provided herein may contain usage of the introductory phrases “at least one” and “one or more” to introduce elements of the methods and structures described herein. However, the use of such phrases should not be construed to imply that the introduction by the indefinite articles “a” or “an” limits the description to embodiments containing only one such recitation. The same holds true for the use of definite articles used to introduce elements.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” and the like include the number recited and refer to ranges which can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

From the foregoing, it will be appreciated that various embodiments of the present disclosure have been described herein for purposes of illustration, and that various modifications may be made without departing from the scope and spirit of the present disclosure. Accordingly, the various embodiments disclosed herein are not intended to be limiting.

This disclosure is accompanied by claims, which are presented only to provide illustrative examples of embodiments described herein. The claims of this patent application are not limiting of embodiments described herein, and instead further describe and support selected embodiments. The scope of such aspects described herein will instead by specified by other claims that might be presented in subsequently-filed non-provisional applications claiming the benefit of the present patent application which claims priority to U.S. Provisional Application No. 62/754,481 filed Nov. 1, 2018, which provisional is incorporated herein by specific reference in its entirety.

All references recited herein are incorporated herein by specific reference in their entirety. This patent application cross-references: U.S. application Ser. No. 15/766,805, which is the U.S. national stage of PCT Application No. PCT/US16/56476 filed Oct. 11, 2016, U.S. Provisional Application No. 62/239,604 filed Oct. 9, 2015, U.S. application Ser. No. 15/919,666 filed Mar. 13, 2018, U.S. application Ser. No. 15/714,984 filed Sep. 25, 2017, and U.S. application Ser. No. 13/842,004 filed Mar. 15, 2013, which are incorporated herein by specific reference in their entirety.

1. A computer-implemented method of identifying genetic biomarkers of a condition, the method comprising:
performing a quantitative image analysis of an image of a phenotype for a condition of a subject for a plurality of subjects with the condition to obtain a first accurate quantitative phenotype;
performing a quantitative genome analysis on each subject of the plurality of subjects or a plurality of different subjects;
obtaining at least one candidate genetic biomarker for the condition in the plurality of subjects from the quantitative image analysis and the quantitative genome analysis;
predicting a clinical response against the at least one candidate genetic biomarker to validate the at least one candidate genetic biomarker;
identifying at least one therapeutic target for the condition based on the at least one candidate genetic biomarker, wherein the at least one therapeutic target for the condition is biologically associated with the at least one candidate genetic biomarker; and
generating a report with the identified at least one therapeutic target for the condition.

2. The computer-implemented method of claim 1, wherein the performing a quantitative genome analysis includes performing a quantitative trait locus genome-wide association study (QTL GWAS).

3. The computer-implemented method of claim 1, further comprising determining a validation metric by predicting a clinical response against the at least one candidate genetic biomarker to validate the at least one candidate genetic biomarker.

4. The computer-implemented method of claim 3, wherein the clinical response prediction is performed to modulate at least one biological pathway associated with the at least one genetic biomarker.

5. The computer-implemented method of claim 4, wherein the predicted clinical response is performed to inhibit at least one biologically active protein of a biological pathway associated with the at least one genetic biomarker.

6. The computer-implemented method of claim 1, further comprising at least one of:
obtaining the images of the phenotype of the subjects, wherein the images accurately illustrate the condition at a given time-point;
obtaining a phenotype accuracy of greater than or about 85% for a quantitative phenotype;
detecting a disease as the condition from the images of the phenotype of the subjects;
defining disease activity across a disease spectrum; or
performing a standardized uptake value ratio (SUVR) analysis with the images of the phenotype of the subject.

7. The computer-implemented method of claim 1, wherein the performing the quantitative image analysis of the image of the phenotype includes at least one of:
calculating a voxel-based amyloid standardized uptake value ratio (SUVR) from the images;
calculating a voxel-based dopamine transporter single photon emission computed tomography (DAT-SPECT) quantitation from the images;
calculating a voxel-based alpha-synuclein ligand binding from the images; or
calculating a regional serotonin receptor ligand binding from the images.

8. The computer-implemented method of claim 1, wherein the images are selected from positron emission tomography (PET) and magnetic resonance imaging (MRI), single photon emission computed tomography (SPECT), and combinations thereof.

9. The computer-implemented method of claim 3, wherein the predicting the clinical response includes at least one of:
performing a hippocampus-masked voxel-based Tau SUVR by genotype quantitation;
performing a determination of memory tracking by a voxel-based Tau SUVR by genotype quantitation with the hippocampus;
performing a voxel-based Tau quantitation within the hippocampus;
performing a Tau imaging analysis that tracks a clinical response; or
performing a clinical response tracking measure.

10. The computer-implemented method of claim 3, wherein the predicting the clinical response includes at least one of:
performing a tremor quantitation; or
performing an automated tremor quantitation,
wherein the tremor quantitation is optionally by genotype.

11. The computer-implemented method of claim 3, wherein the predicting the clinical response includes:
calculating a voxel-based serotonin receptor ligand binding from images.

12. The computer-implemented method of claim 1, further comprising performing a phenotype prediction from a genotype based on the at least one candidate genetic biomarker for the condition.

13. The computer-implemented method of claim 12, wherein the phenotype prediction includes at least one of:
phenotype prediction with deep learning from a single nucleotide polymorphism (SNP) model;
a quantitative phenotype analysis with positron emission tomography (PET) images;
predict voxel-based amyloid SUVRs with deep learning SNP model;
predict voxel-based DAT-SPECT quantitation with deep learning SNP model;
predict voxel-based alpha-synuclein ligand binding with deep learning SNP model; or
predict regional serotonin receptor ligand binding with deep learning SNP model.

14. The computer-implemented method of claim 1, comprising at least one of:
listing the at least one candidate genetic biomarker in the report;
analyzing a biological pathway having the at least one candidate genetic biomarker; or
comparing the at least one candidate genetic biomarker to a biological pathway associated with the condition.

15. The computer-implemented method of claim 1, wherein the condition is:
a syndrome having a set of medical signs and symptoms that are correlated with each other in the subject;
a disease having a pathophysiological response to external or internal factors in the subject; or
a disorder having a disruption to regular bodily structure and/or function in the subject.

16. The computer-implemented method of claim 15, wherein the condition is selected from Alzheimer's disease, Parkinson's disease, and Major depressive disorder (MDD).

17. A system for identifying genetic biomarkers of a condition, the system comprising:
a deep phenotyping unit;
a QTL GWAS analysis unit downstream from the deep phenotyping unit; and
a clinical response validation unit downstream from the QTL GWAS analysis unit.

18. The system of claim 17, wherein components of the system are configured in accordance with one or more of:
the deep phenotyping unit is configured for performing at least one of:
performing an accurate quantitative phenotype analysis;
calculating voxel-based amyloid SUVRs from PET and T1 MRI images;
calculating voxel-based DAT-SPECT quantitation from SPECT and T1 MRI images;
calculating voxel-based alpha-synuclein ligand binding from PET and T1 MRI images; or
calculating regional serotonin receptor ligand binding from PET and T1 MRI images;
the QTL GWAS analysis unit is configured for performing a quantitative trait locus genome-wide association study (QTL GWAS); and
the clinical response validation unit is configured for performing at least one of:
predicting a clinical response against the at least one candidate genetic biomarker to validate the at least one candidate genetic biomarker;
comparing hippocampus-masked voxel-based Tau SUVRs by genotype;
comparing tremor quantitation by genotype; or
calculating voxel-based serotonin receptor ligand binding with PET and T1 MRI images.

19. The system of claim 18, further comprising at least one of:
a gene expression analysis unit configured for performing gene expression profiling, which may or may not be automated;
a pathway mapping unit configured for mapping one or more biological pathways of genes identified by the gene expression analysis unit;
a phenotype prediction unit configured to use a deep learning model to predict the phenotypes identified by the deep phenotyping unit or to predict new phenotypes for new investigations of new conditions;
a pattern and/or trend analysis unit optionally used with the deep phenotyping unit and configured to determine patterns or trends for the phenotype;
a statistical analysis unit configured to perform statistical analyses of any data or metric of the methods;
a text mining plausibility analysis unit configured to determine therapeutic target plausibility by text mining and/or to determine a chemical modulator of a biological entity of a biological pathway associated with the therapeutic target;
a new chemical entity (NCE) prediction unit configured to design or generate an NCE that can modulate the therapeutic target; or
a reporting unit is configured to generate reports.

20. A computer-implemented method of training an artificial neural network for detection of Amyloid positivity or early Alzheimer's Disease comprising:
collecting a set of corresponding positron emission tomography (PET) images, magnetic resonance imaging (MRI) images and corresponding genotype data from a database;
calculating voxel-based amyloid SUVRs from the PET images and T1 MRI images to obtain an accurate quantitative phenotype of early Alzheimer's Disease;
creating a training set comprising said quantitative phenotype and corresponding genotype data; and
training the artificial neural network to predict the quantitative phenotype from genotype data using the training set.

21. The computer-implemented method of claim 20, wherein the artificial neural network is trained using genotype data comprising at least a subset of SNP id 1-32, and optionally SNP id 33 to predict the quantitative phenotype:

| ID | Chr | SNP | A1 | A2 |
|---|---|---|---|---|
| 1 | 1 | rs4912453 | T | C |
| 2 | 1 | rs12120406 | G | A |
| 3 | 1 | rs11161719 | C | T |
| 4 | 1 | rs6576798 | C | T |
| 5 | 3 | rs2030515 | G | A |
| 6 | 4 | rs4689137 | G | C |
| 7 | 4 | rs10007765 | C | G |
| 8 | 4 | rs10029820 | G | A |
| 9 | 6 | rs9458512 | A | G |
| 10 | 7 | rs1001029 | A | G |
| 11 | 7 | rs1001026 | A | G |
| 12 | 7 | rs13222318 | C | T |
| 13 | 7 | rs62444137 | A | G |
| 14 | 7 | rs17680408 | A | G |
| 15 | 7 | rs10234008 | T | C |
| 16 | 7 | rs917321 | T | C |
| 17 | 7 | rs993900 | A | G |
| 18 | 14 | rs8009420 | A | C |
| 19 | 14 | rs213563 | A | G |
| 20 | 15 | rs7164265 | C | T |
| 21 | 15 | rs1551466 | C | G |
| 22 | 15 | rs12916234 | A | C |
| 23 | 15 | rs9920618 | C | T |
| 24 | 16 | rs8056050 | C | T |
| 25 | 18 | rs4891826 | G | T |
| 26 | 19 | rs71352238 | C | T |
| 27 | 19 | rs2075650 | G | A |
| 28 | 19 | rs157582 | A | G |
| 29 | 19 | rs769449 | A | G |
| 30 | 19 | rs56131196 | A | G |
| 31 | 19 | rs4420638 | G | A |
| 32 | 19 | rs17815373 | A | G |
| 33 | 16 | rs4402561 | C | T. |

22. A computer-implemented method comprising:
calculating voxel-based amyloid SUVRs from PET and T1 MRI images;
performing a quantitative trait locus genome-wide association study (QTL GWAS);
obtaining at least one candidate genetic biomarker for the condition in the plurality of subjects from the quantitative image analysis and the quantitative genome analysis;
comparing hippocampus-masked voxel-based Tau SUVRs by genotype;
predicting voxel-based amyloid SUVRs with deep learning SNP model; and
generating a report with the predicted voxel-based amyloid SUVRs.
